SEQUENCE LISTING (8 sequences)

SEQ ID NO: 1 - protein, 972 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 972 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 972 aa, Homo sapiens
STIEEQAKYFLDKFNHEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 972 aa, Homo sapiens
STIEEQAKYFLDYFNVQAEDLFYQSSLASWNYNTNITEENVQNMNNAGDKWSAFLKEQSTYAQRYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 973 aa, Homo sapiens
STIEEQAKYFLDKFNHEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 973 aa, Homo sapiens
STIEEQAKYFLDKFNHEAEDLFYQSSLASWNYNTNITEENVQNMNNAGDKWSAFLKEQSTYAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 973 aa, Homo sapiens
STIEEQAKYFLDYFNVQAEDLFYQSSLASWNYNTNITEENVQNMNNAGDKWSAFLKEQSTYAQRYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 973 aa, Homo sapiens
STIEEQAKYFLDYFNVQAEDLFYQSSLASWNYNTNITEENVQNMNNAGDKWSAFLKEQSTYAQRYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHREMGRIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGGSGGGGSGGGGSEPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0102474 A1
(43) Pub. Date: Apr. 16, 2026

(54) MODIFIED ACE2 PROTEINS WITH IMPROVED ACTIVITY AGAINST SARS-COV-2

(71) Applicants: Deutsches Krebsforschungszentrum Stiftung des öffentlichen Rechts, Heidelberg (DE); Eberhard Karls Universität Tübingen Medizinische Fakultät, Tübingen (DE)

(72) Inventors: Helmut SAHLI, Heidelberg (DE); Latifa ZEKRI, Heidelberg (DE); Gundram JUNG, Rottenburg (DE); Michael SCHINDLER, Reutlingen (DE); Natalia RUETALO BUSCHINGER, Tübingen (DE); Mary CHRISTIE, Heidelberg (DE); Martin PFLÜGLER, Tübingen (DE)

(73) Assignees: Deutsches Krebsforschungszentrum Stiftung des öffentlichen Rechts, Heidelberg (DE); Eberhard Karls Universität Tübingen Medizinische Fakultät, Tübingen (DE)

(21) Appl. No.: 19/116,337

(22) PCT Filed: Sep. 27, 2023
(86) PCT No.: PCT/EP2023/076781

§ 371 Date: Mar. 27, 2025

(30) Foreign Application Priority Data
Sep. 28, 2022 (EP) 22198499.0

(51) Int. Cl. A61K 38/48; A61P 31/14; C12N 9/48

(52) U.S. Cl. CPC A61K 38/4813; A61P 31/14; C12N 9/485; C12Y 304/17023; C07K 2319/30

(57) ABSTRACT

The present invention relates to therapeutic proteins and medical uses thereof. The present invention relates to modified ACE2 protein having increased binding affinity for the SARS-COV-2 spike protein compared to wildtype ACE2 protein, wherein said modified ACE2 protein is lacking enzymatic activity and is fused to (i) a modified Fc domain of human immunoglobulin, wherein the Fc domain has either enhanced immunostimulating activity (Fc⁺) or attenuated immunostimulating activity (Fc⁻) or (ii) a protein that specifically binds to T cells or further enhances immunostimulating activity. The invention also relates to a polynucleotide encoding the modified ACE2 protein of the present invention, a vector or expression construct comprising the polynucleotide, a host cell comprising the polynucleotide or the vector or expression construct, and a non-human transgenic organism comprising the polynucleotide or the vector or expression construct. Moreover, the present invention relates also to a method for the manufacture of a modified ACE protein according to the present invention and to a medicament comprising the modified ACE2 protein, the polynucleotide or the vector or expression construct of the invention. Furthermore, the invention relates to medical uses of the modified ACE2 protein, the polynucleotide or the vector or expression construct in treating and/or preventing a disease or disorder associated with SARS-COV-2 infection. Finally, the invention provides a kit comprising the modified ACE2 protein, the polynucleotide or the vector or expression construct of the invention.

The present invention relates to therapeutic proteins and medical uses thereof. The present invention relates to a modified ACE2 protein having increased binding affinity for the SARS-CoV-2 spike protein compared to wildtype ACE2 protein, wherein said modified ACE2 protein is lacking enzymatic activity and is fused to (i) a modified Fc domain of human immunoglobulin, wherein the Fc domain has either enhanced immunostimulating activity (Fc⁺) or attenuated immunostimulating activity (Fc⁻) or (ii) a protein that specifically binds to T cells or further enhances immunostimulating activity. The invention also relates to a polynucleotide encoding the modified ACE2 protein of the present invention, a vector or expression construct comprising the polynucleotide, a host cell comprising the polynucleotide or the vector or expression construct, and a non-human transgenic organism comprising the polynucleotide or the vector or expression construct. Moreover, the present invention relates also to a method for the manufacture of a modified ACE protein according to the present invention and to a medicament comprising the modified ACE2 protein, the polynucleotide or the vector or expression construct of the invention. Furthermore, the invention relates to medical uses of the modified ACE2 protein, the polynucleotide or the vector or expression construct in treating and/or preventing a disease or disorder associated with SARS-COV-2 infection. Finally, the invention provides a kit comprising the modified ACE2 protein, the polynucleotide or the vector or expression construct of the invention.

In 2020, the severe acute respiratory syndrome coronavirus 2 (SARS-COV-2) causing the Covid-19 disease has become a pandemic due to its high transmissibility and deadly outcome. The infection by severe acute respiratory syndrome coronavirus 2 (SARS-COV-2) is initiated by binding of Spike protein of the virus to host receptor, primarily human angiotensin-converting enzyme 2 (hACE2). Upon binding, fusion of viral and host membranes occurs allowing the virus to enter the host cell and start viral replication.

There have been some reports on neutralizing antibodies. Those antibodies typically bind to the receptor binding domain of the spike protein and thereby inhibit binding to hACE2 and, thus, finally entering of the virus into the host cell (Asamow 2021, Cell 184, 3192-3204; Sharma 2021, Proteins. 2021; 1-11; WO2021/1207433). The antibodies reported so far, have affinities in the nano-molar range.

However, SARS-Cov-2 is rapidly mutating resulting in many novel virus variants of concern and antibodies reported so far, typically, bind to spike proteins of only one virus variant or a small subset of virus variants. Accordingly, a drawback of antibody-based therapeutics is that they are usually variant specific therapeutics.

It has been proposed to use fusion proteins comprising ACE2 rather than antibodies for binding to- and neutralization of viral spike proteins. However, the binding affinity of wildtype human ACE2 to such proteins is usually lower than that of antibodies and the enzymatic activity of ACE2, which is important for regulation of the renin-angiotensin-aldosterone system (RAAS), may lead to undesirable toxicity upon application to humans. Thus, recombinant ACE2 proteins with increased affinity to the original version of the viral spike protein have been developed (Chan et al. 2020; Science 369:1261, Higuchi et al. 2021; Nat. Commun. 12:3802) and the use of ACE2 variants with attenuated enzymatic activity has been suggested (Payandeh et al. 2020; J. Theor. Biol. 505:110425). Those variants are known in the field.

For instance, Ferrari et al. 2021 describes soluble ACE2-based fusion proteins with Fc domains with enhanced activity against SARS-COV-2 variants, wherein the proteins are mutated to remove catalytical activity from the ACE2 part and to abrogate FcγR engagement due to modifications in the Fc domain. Tanaka et al. 2021 describes a method to predict substitutions in ACE2-based constructs that lead to enhanced affinity for SARS-COV-2 variants and improved SARS-COV-2 neutralization capability. Similarly, Svilenov et al. 2021 describes engineered ACE2-IgG4-Fc fusion proteins that avoid Fc-receptor activation, preserve ACE2 enzymatic activity and show promising pharmaceutical properties.

However, there remains a need to develop further SARS-COV-2 therapies having increased neutralization potential in terms of strength and range in order to efficiently and reliably treat and prevent SARS-COV-2 infections. This is even more important given the fact that the global pandemic is producing constantly novel viral variants of concern. As of today, there have been reports for multiple variants, including: SARS-COV-2 alpha variant (B.1.1.7), SARS-COV-2 beta variant (B.1.1351) SARS-COV-2 delta variant (B.1.617.2) or SARS-COV-2 omicron variant (B.1.1.529) including the currently known omicron sub-variants BA.1, BA.2, BA.3, BA.4 and BA.5. Moreover, there is also a need for SARS-COV-2 therapies having reduced side effects, e.g., due to immunological overstimulation.

The technical problem underlying the present invention may be seen as the provision of means and methods for complying with the aforementioned needs. The technical problem is solved by the embodiments characterized in the claims and described herein below.

The present invention relates to a modified ACE2 protein having increased binding affinity for the SARS-COV-2 spike protein compared to wildtype ACE2 protein, wherein said modified ACE2 protein is lacking enzymatic activity and is fused to:
(i) a modified Fc domain of human immunoglobulin, wherein the Fc domain has either enhanced immunostimulating activity (Fc⁺) or attenuated immunostimulating activity (Fc⁻); or
(ii) a protein that specifically binds to T cells or further enhances immunostimulating activity.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The term “about” in the context of the present invention means+/−20%, +/−10%, +/−5%, +/−2% or +/−1% from the indicated parameters or values. This also takes into account usual deviations caused by measurement techniques and the like.

The term “ACE2 protein” as used herein refers to the Angiotensin-converting enzyme 2 (ACE2). ACE2 is an enzyme that can be found either attached to the membrane of cells in the intestines, kidney, testis, gallbladder, and heart or in a soluble form (sACE2). Both membrane bound and soluble ACE2 are integral parts of the renin-angiotensin-aldosterone system (RAAS) that exists to control blood pressure in an organism. Membrane bound Angiotensin-converting enzyme 2 (mACE2) is a zinc-containing metalloenzyme. It contains an N-terminal peptidase M2 domain and a C-terminal collectrin renal amino acid transporter domain and is a single-pass type I membrane protein, with its enzymatically active domain exposed on the surface of cells. The extracellular domain of mACE2 can be cleaved from the transmembrane domain by another enzyme known as ADAM17 a member of the sheddase enzyme family, during the protective phase of RAAS, the Renin Angiotension Aldosterone System which regulates our body's blood pressure. The resulting cleaved protein is known as soluble ACE2 or sACE2. It is released into the bloodstream where one of sACE2's functions is to turn excess angiotensin II into angiotensin 1-7 which binds to MasR receptors creating localized vasodilation and hence decreasing blood pressure.

Membrane ACE2 also serves as the entry point into cells for some coronaviruses, including HCoV-NL63, SARS-COV, and SARS-COV-2. The SARS-COV-2 spike protein itself is known to damage the endothelium via downregulation of ACE2.

The human version of the ACE2 has an amino acid sequence as found, preferably, under accession number Q9BYF1 in the UniProt database. Orthologs from many species have been reported including mice.

The term “wildtype ACE2 protein” as used herein refers to an ACE2 protein that occurs in an organism and that has not been genetically modified. A wildtype ACE2 protein, thus, is typically capable of binding to binding partners such as the SARS-COV-2 spike protein with a normal, i.e. unchanged, binding affinity. Moreover, the wildtype ACE2 protein as referred to in accordance with the present invention, typically, exhibits enzymatic activity, i.e. angiotensin converting activity.

The “modified ACE2 protein” according to the present invention is genetically modified, i.e. it contains in its amino acid sequence at least one amino acid exchange which may affect one or more of its physiological activities.

Preferably, the modified ACE2 protein of the present invention shall exhibit increased binding affinity for the SARS-VoV-2 spike protein and, preferably, for its receptor binding domain (RBD).

The term “increased binding affinity” as used herein refers to a binding affinity which is significantly increased when compared with the binding affinity of the wildtype ACE2 protein for the SARS-COV-2 spike protein and, in particular, its RBD.

The binding affinity may be characterized by the equilibrium dissociation constant (Kd) which indicates the propensity for a complex of two bound molecules to dissociate into its free components, i.e. free SARS-COV-2 spike protein and free ACE2 protein. The equilibrium dissociation constant (Kd) can be expressed as follows:

$$Kd = {[ A ]}_{x} * {[ B ]}_{y} / [ {A}_{x} {B}_{y} ]$$

wherein [Aₓ], [B_{y}], and [AₓB_{y}] are the concentrations of A, B and AB at the equilibrium, respectively. Thus, the smaller the equilibrium dissociation constant, the more tightly bound the ligand is, or the higher the affinity between ligand and protein. The equilibrium dissociation constant referred to in accordance with the present invention can be determined by techniques well known in the art, preferably, it is to be determined using surface plasmon resonance.

The binding affinity can be also characterized by determining EC50 or IC50 values for the binding of two components. The EC50 or IC50 values indicate the concentration where half-maximal binding between the components is reached. EC50 and IC50 values can be typically determined by using ELISA formats or by using FACS analysis. Preferably, the values may be determined as described in the accompanying Examples, below.

Preferably, the binding affinity of the modified ACE2 protein for the SARS-COV-2 spike protein and, preferably, its RBD, is increased compared to the binding affinity of the wildtype ACE2 protein for the said SARS-COV-2 spike protein or its RBD by at least a factor of about 2, about 3, about 4, about 5, about 6, about 8, about 9 or about 10. It will be understood that for a comparison of the binding affinities, the binding affinities shall be typically determined in comparable settings, i.e. if the binding protein of wildtype ACE2 is compared to recombinant SARS-CoC-2 spike protein, the binding affinity for modified ACE2 protein shall also be determined using recombinant SARS-CoC-2 spike protein. Similarly, if the binding affinity is determined for SARS-COV-2 spike protein present on infected cells for the wildtype ACE2 protein, the binding affinity of the modified ACE2 protein shall also be determined vis-à-vis SARS-COV-2 spike protein on infected cells. Moreover, if binding affinity shall be determined for parts of the SARS-COV-2 spike protein such as the RBD, those parts of the spike protein shall be used for determining both, the binding affinity of the wildtype as well as the modified ACE2 protein.

Preferably, said increased binding affinity for the SARS-COV-2 spike protein of the modified ACE2 protein of the invention is increased binding affinity for the receptor binding domain (RBD) of the SARS-COV-2 spike protein.

The term “SARS-COV-2 spike protein” as used herein refers to a glycoprotein that forms a homotrimer at the surface of SARS-COV-2 (Wrapp et al, Science, 367:1260-1263). In the trimeric structure of the spike protein, the RBD may be exhibited by each monomer in either a so-called “up” or a so-called “down” configuration. The structure and amino acid composition of the SARS-COV-2 spike protein is well known in the art, for WT SARS-COV-2 as well as for various variants of the virus, such as SARS-COV-2 alpha variant (B.1.1.7), SARS-COV-2 beta variant (B.1.1351) SARS-COV-2 delta variant (B.1.617.2) or SARS-COV-2 omicron variant (B.1.1.529) including its sub-variants. Sequences for wildtype SARS-COV-2 may be found in BetaCoV/Wuhan/IVDC-HB-01/2019, accession ID: EPI_ISL 402119; BetaCoV/Wuhan/IVDC-HB-04/2020, accession ID: EPI_ISL 402120; BetaCoV/Wuhan/IVDC-HB-05/2019, accession ID: EPI_ISL 402121.

The term “receptor binding domain (RBD)” as used herein refers to a region of the SARS-COV-2 spike protein which is involved in binding of the said spike protein of the virus to the human Angiotensin Converting Enzyme (hACE)-2 receptor on host cells. The RBD consists of amino acids 319 to 541 of the SARS-COV-2 spike protein of WT SARS-COV-2. It will be understood that in virus variants of SARS-COV-2, the position may differ due to the presence of one or more additional amino acids and/or deletions of one or more amino acids. Typically, such variants, however, shall also comprise a RBD which consists of amino acids corresponding to the amino acids of the RBD in WT SARS-COV-2 at amino acid positions 319 to 541.

Preferably, said SARS-COV-2 spike protein is a wildtype SARS-COV-2 spike protein or a variant thereof, preferably, a spike protein of SARS-COV-2 alpha, beta, gamma, delta, kappa or omicron including its sub-variants.

Also preferably, the modified ACE2 protein of the present invention shall lack enzymatic activity. The term “lacking enzymatic activity” as used herein refers to a significant reduction of the enzymatic activity exhibited by a modified ACE2 protein compared to wildtype ACE2 protein. Thus, the term relates to a modified ACE2 protein having either significantly reduced enzymatic activity or even no enzymatic activity. Significantly reduced enzymatic activity as used herein refers to a reduction of the enzymatic activity compared to the activity of the wildtype ACE2 protein of at least at least about 40%, at least about 50%, at least about 60%, at least about 70%, at least about 80%, at least about 90% or at least about 95%. The enzymatic activity can be measured by assays known in the art and, preferably, those described in the accompanying Examples, below. The reduction of enzymatic carboxypeptidase activity can be achieved by modifying and/or deleting the amino acids which participate in catalyzing the conversion of angiotensin, i.e. the cleavage of angiotensin I into the vasoconstricting angiotensin II.

The term “immunoglobulin” as referred to herein relates to an antibody an immunoglobulin from any one of immunoglobulin class A, D, E, G or M. It is also referred to herein as antibody. In particular, it is envisaged that the fragment crystallizable (Fc) domain of the antibody is fused to the modified ACE2 protein of the invention. An Fc domain comprises two heavy chain fragments comprising the CH2 and CH3 domains of an antibody. The two heavy chain fragments are held together by two or more disulfide bonds and by hydrophobic interactions of the CH3 domains. The Fc domain is the tail region of an antibody that interacts with cell surface receptors called Fc receptors and some proteins of the complement system. This property allows antibodies to activate the immune system. The Fc domains may also comprise glycosylation sites. Glycosylation of the Fc domain may be essential for Fc-receptor-mediated immune activity. The other part of an antibody is the so-called Fab domain. It contains variable regions that define the target specificity of an antibody. The modified Fc domain of an immunoglobulin is typically capable of interacting with Fc receptors and proteins of the complement system with a normal, i.e. unchanged, binding affinity. Moreover, the interaction of the Fc domain with Fc receptors or proteins of the complement system, typically, results in Fc-mediated effector functions, such as antibody-dependent cellular cytotoxicity (ADCC), antibody-dependent cellular phagocytosis (ADCP), and complement-dependent cytotoxicity (CDC).

Many modifications in the Fc domain of antibodies have been reported. Some modifications exhibit increased Fc-receptor binding while others result in decreased or loss of Fc-receptor binding, also called “Fc attenuation”.

A modified Fc domain may be fused to various proteins to create fusion proteins with altered immunological effector functions. If fusion proteins contain antibody Fc parts, such Fc parts may support virus elimination by well-established mechanisms, e.g. to the various types of Fc receptors (FcRs) expressed by immunological effector cells or by activation of the complement system initiated by interaction of the Fc part with the complement protein C1q. In general Fc mediated effector functions can be modified by genetic modifications comprising either the introduction of suitable point mutations or an exchange of the entire Ig-isotype, e.g. from IgG to IgA (Saunders et al. 2019). In many cases point mutations are used to enhance the effector function of the Fc part by increasing its binding to various FcRs. Binding to FcRIIa, e.g expressed on antigen presenting cells, appears to be of particular interest since this interaction has been described to elicit a vaccinal effect by enhancing specific immune reactions.

In some cases, Fc/FcR interactions may result in undesired enhancement of viral spread designated antibody dependent enhancement (ADE), which may be caused by uptake of antibody coated viral particles into FcR expressing cells, e.g. alveolar macrophages. It is suggested that severe lung injury in SARS-COV-2 infected subjects is likely to be due to macrophage-induced immune activation, i.e. inflammatory messengers that are released by macrophages leading to severe inflammatory cascades, than direct, virus-induced damage to alveoli. Thus, in cases where alveolar macrophages are already activated (e.g. in a late stage SARS-COV-2 patient), it is preferable to attenuate rather than enhance Fc/FcR interaction by genetic modification. However, in several cases it is preferred to enhance Fc/FcR interactions, for example, in early stage SARS-COV-2 infections, when alveolar macrophages have not been activated.

A modified Fc domain that has enhanced immunostimulating activity according to the present invention refers to modified Fc domain variants that significantly stimulate the immune system by inducing activation or increasing activity of any of its components. In particular, enhanced immunostimulation means that the Fc domain exhibits significantly increased Fc-mediated effector functions, i.e. ADCC, ADCP or CDC compared to a unmodified (wildtype) Fc domain. The term “enhanced immunostimulating activity” herein is also meant that a modified Fc domain variant binds to an Fc receptor with a significantly higher equilibrium constant of association (K_{A} or Kₐ) or lower equilibrium constant of dissociation (K_{D} or K_{d}) than the unmodified Fc domain variant.

In certain situations, such as in early stage SARS-COV-2 infections, when alveolar macrophages have not been activated, it shall be preferred to use a modified ACE2 protein having a modified Fc domain that has enhanced immunostimulating activity. Such a modified ACE2 protein shall be particularly efficient to treat and/or prevent SARS-COV-2 infection in a subject due to its strong immunostimulating activity and can be applied without the risk of side effects caused by stimulation of the non-activated alveolar macrophages.

Preferably, increased Fc receptor binding can be achieved by introducing the Fc domain mutants of human IgG1 referred to herein generally as “SDIE”, which denote the mutations S239D/I332E.

Means for increasing ADCC activity through specific Fc region mutations include the Fc variants comprising at least one amino acid substitution at a position selected from the group consisting of: 234, 235, 239, 240, 241, 243, 244, 245, 247, 262, 263, 264, 265, 266, 267, 269, 296, 297, 298, 299, 313, 325, 327, 328, 329, 330 and 332, wherein the numbering of the residues in the Fc region is that of the EU index as in Kabat (Kabat 1987, Sequences of Proteins of Immunological Interest (National Institute of Health, Bethesda, Md. 1987). More specifically, said Fc variants comprise at least one substitution selected from the group consisting of L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L, F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, E269H, E269Y, E269F, E269R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, N325Q, N325L, N325I, N325D, N325E, N325A, N325T, N325V, N325H, A327N, A327L, L328M, L328D, L328E, L328N, L328Q, L328F, L328I, L328V, L328T, L328H, L328A, P329F, A330L, A330Y, A330V, A330I, A330F, A330R, A330H, I332D, I332E, I332N, I332Q, I332T, I332H, I332Y and I332A, wherein the numbering of the residues in the Fc region is that of the EU index as in Kabat. Moreover, Fc variants can also be, preferably, selected from the group consisting of V264L, V264I, F241W, F241L, F243W, F243L, F241L/F243L/V262I/V264I, F241W/F243W, F241W/F243W/V262A/V264A, F241L/V262I, F243L/V264I, F243L/V262I/V264W, F241Y/F243Y/V262T/V264T, F241E/F243R/V262E/V264R, F241E/F243Q/V262T/V264E, F241R/F243Q/V262T/V264R, F241E/F243Y/V262T/V264R, L328M, L328E, L328F, I332E, L3238M/I332E, P244H, P245A, P247V, W313F, P244H/P245A/P247V, P247G, V264I/I332E, F241E/F243R/V262E/V264R/I332E, F241E/F243Q/V262T/264E/I332E, F241R/F243Q/V262T/V264R/I332E, F241E/F243Y/V262T/V264R/I332E, S298A/I332E, S239E/I332E, S239Q/I332E, S239E, D265G, D265N, S239E/D265G, S239E/D265N, S239E/D265Q, Y296E, Y296Q, T299I, A327N, S267Q/A327S, S267L/A327S, A327L, P329F, A330L, A330Y, I332D, N297S, N297D, N297S/I332E, N297D/I332E, N297E/I332E, D265Y/N297D/I332E, D265Y/N297D/T299L/I332E, D265F/N297E/I332E, L328I/I332E, L328Q/I332E, I332N, I332Q, V264T, V264F, V240I, V263I, V266I, T299A, T299S, T299V, N325Q, N325L, N325I, S239D, S239N, S239F, S239D/I332D, S239D/I332E, S239D/I332N, S239D/I332Q, S239E/I332D, S239E/I332N, S239E/I332Q, S239N/I332D, S239N/I332E, S239N/I332N, S239N/I332Q, S239Q/I332D, S239Q/I332N, S239Q/I332Q, Y296D, Y296N, F241Y/F243Y/V262T/V264T/N297D/I332E, A330Y/I332E, V264I/A330Y/I332E, A330L/I332E, V264I/A330L/I332E, L234D, L234E, L234N, L234Q, L234T, L234H, L234Y, L234I, L234V, L234F, L235D, L235S, L235N, L235Q, L235T, L235H, L235Y, L235I, L235V, L235F, S239T, S239H, S239Y, V240A, V240T, V240M, V263A, V263T, V263M, V264M, V264Y, V266A, V266T, V266M, E269H, E269Y, E269F, E269R, Y296S, Y296T, Y296L, Y296I, A298H, T299H, A330V, A330I, A330F, A330R, A330H, N325D, N325E, N325A, N325T, N325V, N325H, L328D/I332E, L328E/I332E, L328N/I332E, L328Q/I332E, L328V/I332E, L328T/I332E, L328H/I332E, L328I/I332E, L328A, I332T, I332H, I332Y, I332A, S239E/V264I/I332E, S239Q/V264I/I332E, S239E/V264I/A330Y/I332E, S239E/V264I/S298A/A330Y/I332E, S239D/N297D/I332E, S239E/N297D/I332E, S239D/D265V/N297D/I332E, S239D/D265I/N297D/I332E, S239D/D265L/N297D/I332E, S239D/D265F/N297D/I332E, S239D/D265Y/N297D/I332E, S239D/D265H/N297D/I332E, S239D/D265T/N297D/I332E, V264E/N297D/I332E, Y296D/N297D/I332E, Y296E/N297D/I332E, Y296N/N297D/I332E, Y296Q/N297D/I332E, Y296H/N297D/I332E, Y296T/N297D/I332E, N297D/T299V/I332E, N297D/T299I/I332E, N297D/T299L/I332E, N297D/T299F/I332E, N297D/T299H/I332E, N297D/T299E/I332E, N297D/A330Y/I332E, N297D/S298A/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, and S239D/264I/A330L/I332E, wherein the numbering of the residues in the Fc domain is that of the EU index as in Kabat (see WO2004/029207).

A modified Fc domain that has attenuated immunostimulating activity according to the present invention refers to modified Fc domain variants that significantly attenuate or abrogate the immune system, i.e. the modified Fc domain variants induce a weakened activation of immune system components or do not induce an activation at all. In particular, attenuated immunostimulation means that the Fc domain exhibits significantly reduced or abrogated Fc-mediated effector functions, i.e. ADCC, ADCP or CDC. The term “attenuated effector function” herein is also meant that a modified Fc domain variant binds to an Fc receptor with a significantly lower equilibrium constant of association (K_{A} or Kₐ) or higher equilibrium constant of dissociation (K_{D} or K_{d}) than the unmodified Fc domain variant.

In certain situations, such as in late stage SARS-COV-2 infections, when alveolar macrophages have been activated, it shall be preferred to use a modified ACE2 protein having a modified Fc domain that has attenuated immunostimulating activity. Such a modified ACE2 protein shall be particularly efficient to treat and/or prevent SARS-COV-2 infection in a subject having activated alveolar macrophages since the risk of side effects caused by additional stimulation of alveolar macrophages shall be reduced.

There are well-known modifications that achieve functional Fc abrogation including point mutations and the use of the human IgG4 isotype that lacks binding to most FcRs (Saunders et al. 2019).

For decreasing Fc receptor binding, mutations on, adjacent, or close to sites in the hinge link region (e.g., replacing residues 234, 235, 236 and/or 237 with another residue) can be made, in all of the isotypes, particularly Fc-gamma-RI receptor (see U.S. Pat. No. 6,624,821). Preferably, positions 234, 236 and/or 237 are substituted with alanine and position 235 with glutamate (see U.S. Pat. No. 5,624,821). Position 236 is missing in the human IgG2 isotype. Exemplary segments of amino acids for positions 234, 235 and 237 for human IgG2 are Ala Ala Gly, Val Ala Ala, Ala Ala Ala, Val Glu Ala, and Ala Glu Ala. A preferred combination of mutants is L234A, L235E and G237A, or is L234A, L235A, and G237A for human isotype IgG1. Other substitutions that decrease binding to Fc-gamma receptors are an E233P mutation (particularly in mouse IgG1) and D265A (particularly in mouse IgG2a). Other examples of mutations and combinations of mutations reducing Fc and/or C1q binding are E318A/K320A/R322A (particularly in mouse IgG1), L235A/E318A/K320A/K322A (particularly in mouse IgG2a). Similarly, residue 241 (Ser) in human IgG4 can be replaced, e.g., with proline to disrupt Fc binding.

Disruption of the glycosylation site of human IgG1 can reduce Fc-receptor binding and thereby also affects effector function of the antibody (see WO2006/036291). The tripeptide sequences NXS and NXT, where X is any amino acid other than proline, are the enzymatic recognition sites for glycosylation of the N residue. Disruption of any of the tripeptide amino acids, particularly in the CH2 region of IgG, will prevent glycosylation at that site. For example, mutation of N297 of human IgG1 prevents glycosylation and reduces Fc receptor binding to the antibody.

The Fc-attenuation may be, preferably, achieved by deleting and/or substituting at least one amino acid in the CH2 domain that are able to mediate binding to an Fc-receptor. Typically, at least one amino acid of the hinge region or the CH2 domain that is able to mediate binding to Fc receptors and that is lacking or substituted is, preferably, found at a position selected from the group consisting of sequence position 228, 230, 231, 232, 233, 234, 235, 236, 237, 238, 265, 297, 327, and 330 (numbering of sequence positions according to the EU-index). More specifically, it may be at least one mutation selected from the group consisting of a deletion of amino acid 228, a deletion of amino acid 229, a deletion of amino acid 230, a deletion of amino acid 231, a deletion of amino acid 232, a deletion of amino acid 233, a substitution Glu233 to Pro, a substitution Leu234 to Val, a deletion of amino acid 234, a substitution Leu235 to Ala, a deletion of amino acid 235, a deletion of amino acid 236, a deletion of amino acid 237, a deletion of amino acid 238, a substitution Asp265 to Gly, a substitution Asn297 to Gln, a substitution Ala327 to Gln, and a substitution Ala330 to Ser (numbering of sequence positions according to the EU-index).

Fc silencing and half time optimized Fc domains may be obtained, preferably, by combining the so-called LALA Fc mutant and YTE mutants. Both modifications are well known in the art and for example in detail explained in Saunders et al. 2019. In principle, the mutations are found in the human CH domains. YTE is characterized by the mutations Met252Tyr/Ser254Thr/Thr256Glu. The LALA modification is Leu234Ala/Leu235Ala.

Preferably, the modified ACE2 protein of the invention comprises an amino acid sequence selected from the group consisting of:
a) an amino acid sequence as shown in SEQ ID NO: 3, 4, 6 or 8;
b) an amino acid sequence having at least 70% sequence identity to the amino acid sequence shown in SEQ ID NO: 3, 4, 6 or 8;
c) an amino acid sequence of b) which has in its amino acid sequence the following amino acid exchanges: T27Y, L79Y, N330Y; and
d) an amino acid sequence of b) or c) which has in its amino acid sequence at least one of the following further amino acid exchanges: K31Y, H34V, E35Q, or M82R.

More preferably, the modified ACE2 protein of the invention comprises an amino acid sequence selected from the group consisting of SEQ ID NO: 4 or 8. Most preferably, the modified ACE2 protein of the invention comprises the amino acid sequence of SEQ ID NO: 4.

More preferably, such a variant amino acid sequence is at least 70%, at least 75%, at least 80%, at least 85%, at least 90%, at least 95%, at least 96%, at least 97%, at least 98%, at least 99% identical to the concrete amino acid sequence identified by a SEQ ID No. Sequence identity between two amino acid sequences as referred to herein, in general, can be determined by alignment of two sequences either over the entire length of one of the sequences or within a comparison window. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment and calculation of sequence identity can be done by using published techniques or methods codified in computer programs such as, for example, BLASTP, BLASTN or FASTA. The percent sequence identity values are, preferably, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp or the programs Gap and BestFit, which are part of the GCG software packet (Genetics Computer Group, US), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

There are additional ways of enhancing the immunostimulatory activity of Fc-parts, that is, the c-terminal fusion of proteins that recruit T cells or other immunological effector cells. For example, the modified ACE2 protein of the invention may be fused to antibodies binding to the antigen specific T cell receptor (TCR)/CD3 complex and/or to costimulatory molecules such as CD28, 4-1BB or Ox40 (Riethmüller G 2012; Cancer Immun 12: 12). Antibodies directed to such molecules are known to induce and maintain, respectively powerful T cell activation and thus substantially strengthen an immune response. In addition, cytokines, in particular, IL-15 or modified versions thereof may enhance the activity of immune cells expressing the IL15 receptor, such as CD8+ T cells and NK cells, and may therefore amplify specific immune reactions against target cells.

Usually the targeting part of such recombinant constructs is an antibody and fusion of T cell stimulatory antibodies and cytokines to a targeting antibody results in T cell recruiting bispecific antibodies (bsAbs) or immunocytokines (Neri D. 2019; Cancer Immun Res 7:348), respectively. In the case of T cell stimulating bsAbs, Fc/FcR interactions would have to be prevented to avoid off-target T cell activation by FcR expressing cells. It is pertinent that the targeting part of an immune enhancing construct of the type described could also be a recombinant protein such as ACE2.

The term “a protein that specifically binds to T cells or further enhances immunostimulating activity” as used herein refers to a protein such as antibody that binds to the antigen specific T cell receptor (TCR)/CD3 complex and/or to costimulatory molecules such as CD28, 4-1BB or Ox40 or a cytokine that binds to a cytokine on a T cell such as IL-15 or a modified IL-15 that is capable of binding specifically to a surface molecule on a T cell and as a result of said specific binding is capable of recruiting said T cell. However IL-15, as well as other cytokines, will also recruit effector cells apart from T cells, such as NK cells, monocytes and antigen presenting cells. Preferably, the said protein that specifically binds to T cells or further enhances immunostimulating activity may also comprise a modified Fc domain of human immunoglobulin, wherein the Fc domain has either enhanced immunostimulating activity (Fc+) or attenuated immunostimulating activity (Fc−) as defined elsewhere herein or an unmodified Fc domain.

Advantageously, it has been found in the studies underlying the present invention that a modified ACE2 protein according to the present invention is capable of binding to SARS-COV-2 spike protein with increased affinity and, thereby interferes with the interaction of said protein and the (unmodified) wildtype ACE2 protein on viral target cells. Thus, by administering the modified ACE2 protein of the invention to a subject, viral entry into a target cell shall be blocked since the viral SARS-COV-2 spike proteins are bound to the modified ACE2 proteins. It is advantageous that the modified ACE2 protein has a higher affinity to SARS-CoV2 spike protein compared to wildtype ACE2 protein. Moreover, it is advantageous to inactivate the enzymatic activity of the ACE2 protein in order to avoid undesired side-reactions. By binding to viral particles the fusion protein may prevent entry of the particles into cells and thus viral spread.

Thanks to the present invention, an efficient therapy of SARS-CoV2 infection and diseases associated therewith shall be possible. Moreover, the modified ACE2 protein according of the present invention may also be useful for the prevention of an infection. It has been found in the studies underlying the present invention that, advantageously, the modified ACE2 protein of the invention shall bind with essentially the same or even higher affinity to SARS-CoV2 spike proteins of newly emerged virus variants compared to that of the original virus strain. In marked contrast, most highly specific antibodies developed against the spike protein of the original SARS-CoV2Virus are typically high affinity binders for this particular spike protein and fail to bind to most viral variants. By using modified Fc domains, i.e. Fc⁺ or Fc⁻ domains, or the proteins that specifically bind to T cells, different stages of the disease accompanying the infection may be addressed.

The explanations and definitions made above shall apply mutatis mutandis for all other embodiments described herein except if specified otherwise.

The invention also relates to a polynucleotide encoding the modified ACE2 protein of the present invention.

The term “polynucleotide” as used in accordance with the present invention refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). The term as used herein encompasses the sequence specified herein as well as the complementary or reverse-complementary sequence thereof. Preferably, the polynucleotide is RNA or DNA. The term also encompasses DNAs or RNAs with backbones modified for stability or for other reasons. Moreover, DNAs or RNAs comprising unusual bases, such as inosine, or modified bases, such as tritylated bases, to name just two examples, are also encompassed as polynucleotides. It will be appreciated that a great variety of modifications have been made to DNA and RNA that serve many useful purposes known to those of skill in the art. Every nucleic acid sequence herein that encodes a certain polypeptide of the invention may due to the degeneracy of the genetic code have silent variations. The degeneracy of the genetic code yields a large number of functionally identical polynucleotides that encode the same polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations.

The polynucleotide of the invention shall encode the modified ACE2 protein of the invention, i.e. it shall comprise a nucleic acid sequences which encodes said modified ACE2 protein of the invention. In addition, the polynucleotide of the present invention may comprise additional nucleic acid sequences. Preferably, the polynucleotide of the present invention may comprise in addition to an open reading frame further untranslated sequence at the 3′ and at the 5′ terminus of the coding gene region: at least 500, preferably 200, more preferably 100 nucleotides of the sequence upstream of the 5′ terminus of the coding region and at least 100, preferably 50, more preferably 20 nucleotides of the sequence downstream of the 3′ terminus of the coding gene region.

The polynucleotide of the present invention shall be provided, preferably, either as an isolated polynucleotide (i.e. purified or at least isolated from its natural context such as its natural gene locus) or in genetically modified or exogenously (i.e. artificially) manipulated form. An isolated polynucleotide can, for example, comprise less than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived. The polynucleotide, preferably, is provided in the form of double or single stranded molecule. It will be understood that the present invention by referring to any of the aforementioned polynucleotides of the invention also refers to complementary or reverse complementary strands of the specific sequences or variants there-of referred to before. The polynucleotide encompasses DNA, including cDNA and genomic DNA, or RNA polynucleotides.

Moreover, comprised are also chemically modified polynucleotides including naturally occurring modified polynucleotides such as glycosylated or methylated polynucleotides or artificial modified ones such as biotinylated polynucleotides.

Preferably, the polynucleotide of the present invention has a nucleotide sequence as shown in SEQ ID NO: 2. However, it will be understood that a polynucleotide of the present invention may also have a variant nucleotide sequence that comprise at least one nucleotide deletion, addition and/or substitution compared to the nucleotide sequences shown in the aforementioned SEQ ID NO. Such a variant polynucleotide, however, shall still encode a modified ACE2 protein having the biological functions and properties referred to above. The polynucleotide of the present invention may, thus, have an nucleotide sequence being at least about 70%, at least about 80%, at least about 90%, at least about 95%, at least about 98% or at least about 99% identical to the nucleotide sequence shown in SEQ ID NO: 2. Nucleotide sequence identity can be determined, preferably, by methods well known in the art and referred to elsewhere herein. More typically, nucleotide sequence identity may be determined by aligning two nucleic acid sequence such that the highest degree of matches is achieved. This can be done by using well known algorithms of Altschul or Needelman & Wunsch or Smith & Waterman or those implemented in the BLASTN, FASTA, GCG, GAP, BestFit or Pileup programs. An alignment of two nucleic acid sequences may be a global alignment, i.e. an alignment over the entire range of the nucleotide sequences to be compared or a local alignment, i.e. an alignment over a stretch nucleotides of significant length in both sequences. Typically, the standard settings of the aforementioned programs shall be used for carrying out comparison. Moreover, the polynucleotide of the present invention may also be a polynucleotide encoding a modified ACE2 protein having the biological functions and properties referred to above that is capable of hybridizing under stringent hybridization conditions to the polynucleotide having a nucleotide sequence as shown in SEQ ID NO: 2. Stringent hybridization conditions as referred to in accordance with the present invention are described in standard text books such as Sambrook et al. (Molecular Cloning: A Laboratory Manual, 2nd ed., Cold Spring Harbor Laboratory, Cold Spring Harbor Laboratory Press, Cold Spring Harbor, NY, 1989), and well known to the skilled artisan. Preferably, stringent hybridization conditions are hybridization in 6× sodium chloride/sodium citrate (SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer.

The present invention also contemplates a vector or expression construct comprising the polynucleotide of the present invention.

The term “vector”, preferably, encompasses phage, plasmid, cosmids, viral vectors as well as artificial chromosomes, such as bacterial or yeast artificial chromosomes (YAC). The vector encompassing the polynucleotide of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, f-mating, natural competence, carbon-based clusters, chemically mediated transfer, electroporation or particle bombardment. Suitable methods for the transformation or transfection of host cells, including plant cells, can be found in standard text books such as Sambrook et al. (Molecular Cloning: A Laboratory Manual, 2nd ed., Cold Spring Harbor Laboratory, Cold Spring Harbor Laboratory Press, Cold Spring Harbor, NY, 1989). Alternatively, a plasmid vector may be introduced by heat shock or electroporation techniques. Should the vector be a virus, it may be packaged in vitro using an appropriate packaging cell line prior to application to host cells.

Preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. Suitable expression vectors are known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX, pMAL (New England Biolabs, Beverly, MA) and pRIT5 (Pharmacia, Piscataway, NJ), where glutathione S transferase (GST), maltose E-binding protein and protein A, respectively, are fused with the recombinant target protein. Examples of suitable inducible non-fusion E. coli expression vectors are, inter alia, pTrc and pET 11d. The tar-get gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a co-expressed viral RNA polymerase (T7 gn1). This viral polymerase is provided by the host strains BL21 (DE3) or HMS174 (DE3) from a resident lambda-prophage which harbors a T7 gn1 gene under the transcriptional control of the lacUV 5 promoter. The skilled worker is familiar with other vectors which are suitable in prokaryotic organisms; these vectors are, for example, in E. coli, pLG338, pACYC184, the pBR series such as pBR322, the pUC series such as pUC18 or pUC19, the M113mp series, pKC30, pRep4, pHS1, pHS2, pPLc236, pMBL24, pLG200, pUR290, pIN-III113-B1, lambdagt11 or pBdCl, in Streptomyces plJ101, plJ364, plJ702 or plJ361, in Bacillus pUB110, pC194 or pBD214, in Corynebacterium pSA77 or pAJ667. Examples of vectors for expression in the yeast S. cerevisiae comprise pYep Sec1, pMFa, pJRY88 and pYES2 (Invitrogen Corporation, San Diego, CA). Vectors and processes for the construction of vectors which are suitable for use in other fungi, such as the filamentous fungi, comprise those which are described in detail in standard text books such as van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells, e.g., Sf9 cells, comprise the pAc series and the pVL series.

Yet the vector may be an integration vector. An integration vector refers to a DNA molecule, linear or circular, that can be incorporated, e.g., into a microorganism's genome, such as a bacteria's genome, and provides for stable inheritance of a gene encoding a polypeptide of interest, such as the alcohol acyl transferase of the invention. The integration vector generally comprises one or more segments comprising a gene sequence encoding a polypeptide of interest under the control of additional nucleic acid segments that provide for its transcription.

Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell of the invention can be incorporated into (expression) vectors. For example, a DNA sequence for a signal peptide leader can be fused in-frame to a nucleic acid of the invention so that the alcohol acyl transferase of the invention is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. It is possible to provide a fusion of an additional peptide sequence at the amino or carboxyl terminal end of the polypeptide.

The term “expression construct” as used herein refers to polynucleotides comprising the polynucleotide of the invention and additional functional nucleic acid sequences. An expression construct according to the present invention is, preferably, a linear DNA molecule. Typically, an expression construct in accordance with the present invention may be a targeting construct which allows for random or site-directed integration of the targeting construct into genomic DNA. Such target constructs, preferably, comprise DNA of sufficient length for either homologous or heterologous recombination as described in detail below. In both cases, the construct must be, preferably, impeccable, with structures to control gene expression, such as a promoter, a site of transcription initiation, a site of polyadenylation, and a site of transcription termination. Moreover, it will be understood that an expression construct in accordance with the present invention may also be generated by using genomic modification techniques such as genome editing using the CRISPR/Cas technology.

Yet, the invention contemplates a host cell comprising the polynucleotide or the vector or expression construct of the present invention.

The host cell of the invention is capable of expressing the polypeptide of the invention comprised in the vector or expression construct of the invention. The host cell is, typically transformed or transduced with said vector or expression construct such that the polypeptide of the invention can be expressed from the vector or expression construct. The transformed vector or expression construct may be maintained as a non-integrated vector, for example, a plasmid, or alternatively, may be integrated into the host cell genome as specified elsewhere herein in more detail. A host cell according to the invention may be produced based on standard genetic and molecular biology techniques that are generally known in the art, e.g., as described in standard text books such as Sambrook, J., and Russell, D. W. “Molecular Cloning: A Laboratory Manual” 3d ed., Cold Spring Harbor Laboratory Press, Cold Spring Harbor, NY, (2001); and F. M. Ausubel et al, eds., “Current protocols in molecular biology”, John Wiley and Sons, Inc., New York (1987), and later supplements thereto.

Preferably, said host cell is a bacterial cell, a fungal cell, an animal cell or a plant cell.

Bacterial cells may be gram-positive or gram-negative bacterial cells. Preferred bacterial cells may be selected from the genera Escherichia, Klebsiella, Helicobacter, Bacillus, Lactobacillus, Streptococcus, Amycolatopsis, Rhodobacter, Pseudomonas, Paracoccus, Lactococcus or Pantoea. More preferably, useful gram positive bacterial host cells may be Bacillus alkalophius, Bacillus amyloliquefaciens, Bacillus brevis, Bacillus circulans, Bacillus clausii, Bacillus coagulans, Bacillus firmus, Bacillus jautus, Bacillus lentus, Bacillus licheniformis, Bacillus megaterium, Bacillus pumilus, Bacillus stearothermophilus, Bacillus subtilis, Bacillus thuringiensis, Streptomyces spheroides, Streptomyces thermoviolaceus, Streptomyces lividans, Streptomyces murinus, Streptoverticillum verticillium ssp. verticillium. Rhodobacter sphaeroides, Rhodomonas palustri, or Streptococcus lactis. Also more preferably, useful gram negative bacterial host cells may be Escherichia coli, Pseudomonas sp., preferably, Pseudomonas purrocinia, Pseudomonas fluorescens, Rhodobacter capsulatus, Rhodobacter sphaeroides, Paracoccus carotinifaciens, Paracoccus zeaxanthinifaciens or Pantoea ananatis.

Preferred fungal host cells may be Aspergillus, Fusarium, Trichoderma, Yeast, Pichia, or Saccharomyces host cells. Yeast as used herein includes ascosporogenous yeast, basidiosporogenous yeast, and yeast belonging to the Blastomycetes.

Preferred animal host cells may comprise mammalian host cells, avian host cells, reptilian host cells or insect host cells. Preferred animal host cells are Hela cells, HEK293T, F or E cells, U2OS cells, A549 cells, HT1080 cells, CAD cells, P19 cells, NIH3T3 cells, L929 cells, N2a cells, CHO cells, MCF-7 cells, Y79 cells, SO-Rb50 cells, HepG2 cells, DUKX-X11 cells, J558L cells or BHK cells.

Preferred plant host cells comprise tobacco, rice, wheat, pea or tomato cells.

The present invention relates to a non-human transgenic organism comprising the polynucleotide or the vector or expression construct of the present invention.

The term “non-human transgenic organism” as used herein refers to an organism which has been genetically modified in order to comprise the polynucleotide, vector or expression construct of the present invention. Said genetic modification may be the result of any kind of homologous or heterologous recombination event, mutagenesis or gene editing process. Accordingly, the transgenic non-human organism shall differ from its non-transgenic counterpart in that it comprises the non-naturally occurring (i.e. heterologous) polynucleotide, vector or expression construct in its genome. Non-human organisms envisaged as transgenic non-human organisms in accordance with the present invention are, preferably, multi-cellular organisms, such as an animal, plant, multi-cellular fungi or algae. Preferably, said non-human organism is an animal or a plant. Preferred animals are mammals, in particular, laboratory animals such as rodents, e.g., mice, rats, rabbits or the like, or farming animals such as sheep, goat, cows, horses or the like. Preferred plants are crop plants or vegetables, in particular, selected from the group consisting of tobacco, rice, wheat, pea and tomato. Methods for the production of transgenic non-human organisms are well known in the art; see, standard text books, e.g. Lee-Yoon Low et al., Transgenic Plants: Gene constructs, vector and transformation method. 2018. DOI.10.5772/intechopen. 79369; Pinkert, C. A. (ed.) 1994. Transgenic animal technology: A laboratory handbook. Academic Press, Inc., San Diego, Calif.; Monastersky G. M. and Robl, J. M. (ed.) (1995) Strategies in Transgenic Animal Science. ASM Press. Washington D.C); Sambrook, loc.cit, Ausubel, loc.cit).

The present invention relates also to a method for the manufacture of a modified ACE protein according to the present invention comprising the steps of:
(a) expressing the polynucleotide or the vector or expression construct of the invention in a host cell; and
(b) obtaining the modified ACE2 protein from said host cell.

The term “manufacture” as used herein refers to the process of recombinant production of the modified ACE protein of the invention in a host cell. The manufacture may also comprise further steps such as purifying the produced protein or formulating the said protein or purified protein as a pharmaceutical composition. Accordingly, the aforementioned method of the present invention may consist of the aforementioned steps or may comprise further additional steps.

Expressing the polynucleotide or the vector or expression construct of the invention in a host cell may, for example, also include the step of generating the polynucleotide or vector of the invention as well as the step of introducing said polynucleotide or vector or expression construct into the host cell.

Introducing the polynucleotide or vector or expression construct into a host cell for expression can be done by all techniques available in the art, including salt-based transfection, lipofection, electroporation, injection, viral transfection techniques and the like. The polynucleotide or vector or expression construct may be stably integrated into the genome of the host cell or may be transiently present.

Obtaining the modified ACE2 protein of the invention from the host cell can be achieved by purifying or partially purifying the said protein from the host cells or host cell culture. For protein purification, various techniques may be used including precipitation, filtration, ultra-filtration, extraction, chromatography techniques such as ion-exchange-, affinity- and/or size exclusion chromatography, HPLC or electrophoresis. The skilled person is well aware of how an antibody may be purified in order to provide it in isolated form. Preferred techniques are those described in the accompanying Examples below.

The present invention relates to a medicament comprising the modified ACE2 protein, the polynucleotide or the vector or expression construct of the invention.

The term “medicament” as used herein refers to modified ACE2 protein, polynucleotide, vector or expression construct of the invention said is formulated in a pharmaceutical acceptable manner for pharmaceutical uses. Such a medicament is, preferably, for topical or systemic administration. Conventionally a medicament will be administered intra-muscularly or subcutaneously. However, depending on the nature and the desired therapeutic effect and the mode of action, the medicament may be administered by other routes as well. In particular, in accordance with the present invention, aerosol formulations or sprays applying medicament in the respiratory systems such as the nasal tract or the lung are also conceivable. The medicament is, preferably, administered in conventional dosage forms prepared by combining the ingredients with standard pharmaceutical carriers according to conventional procedures. These procedures may involve mixing or dissolving the ingredients as appropriate to the desired preparation. Preferably, a solution is envisaged for the medicament. It will be appreciated that the form and character of the pharmaceutical acceptable carrier is dictated by the amount of active ingredient with which it is to be combined, the route of administration and other well-known variables. A carrier must be acceptable in the sense of being compatible with the other ingredients of the formulation and being not deleterious to the recipient thereof. The pharmaceutical carrier employed may include a solid, a gel, or a liquid. Examples for solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, are distilled water, physiological saline, Ringer's solutions, dextrose solution, and Hank's solution, and the like. Similarly, the carrier may include time delay material well known to the art, such as glyceryl mono-stearate or glyceryl distearate alone or with a wax. For polynucleotides or vectors, liposomal carriers or genetically engineered viruses may be considered as well. In particular, if a long-term application of the antibody is envisaged, a genetically engineered virus may be administered that produces the antibody of the invention over a long period within an organism to be treated. Said suitable carriers comprise those mentioned above and others well known in the art, see, e.g., standard text books such as Remington's Pharmaceutical Sciences, Mack Publishing Company, Easton, Pennsylvania. In addition, the medicament may also include other carriers, adjuvants, or non-toxic, non-therapeutic, non-immunogenic stabilizers and the like. It is to be understood that the formulation of a medicament takes place under GMP standardized conditions or the like in order to ensure quality, pharmaceutical security, and effectiveness of the medicament.

A therapeutically effective dosage of the modified ACE2 protein, polynucleotide, vector or expression construct of the invention refers to an amount of said compounds to be used in medicament. A therapeutically effective dosage is an amount of a compound that prevents, ameliorates or treats the symptoms accompanying a disease or condition referred to in this specification. Therapeutic efficacy and toxicity of the compound can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and other clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. The medicament referred to herein is administered at least once in order to treat or ameliorate or prevent a disease or condition recited in this specification. However, the said medicament may be administered more than one time.

The invention further contemplates a modified ACE2 protein, a polynucleotide or a vector or expression construct as defined in accordance with the present invention for use in treating and/or preventing a disease or disorder associated with SARS-COV-2 infection.

The term “treating” as used herein refers to any improvement, cure or amelioration of the disease or condition as referred to herein. It will be understood that treatment may not occur in 100% of the subjects to which the antibody has been administered. The term, however, requires that the treatment occurs in a statistically significant portion of subjects (e.g. a cohort in a cohort study). Whether a portion is statistically significant can be determined without further ado by a person skilled in the art using various well-known statistic evaluation tools, e.g., determination of confidence intervals, p-value determination, Student's t-test, Mann-Whitney-U test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.05, 0.01, 0.005, 0.001, or 0.0001.

The term “preventing” as used herein refers to significantly reducing the likelihood with which the disease or condition develops in a subject within a defined window (prevention window) starting from the administration of the antibody onwards. Typically, the prevention window is within 1 to 5 days, within 1 to 3 weeks, within 1 to 3 months or within 3 to 6 months or 3 to 12 months. However, it will be understood that the preventive window may, dependent on the kind of medicament, also be several years up to the entire life time. The prevention window depends on the amount of antibody, polynucleotide or vector which is administered and the applied dosage regimen. Typically, suitable prevention windows can be determined by the clinician based on the amount of antibody or polynucleotide to be administered and the dosage regimen to be applied without further ado. It will be understood that prevention may not occur in 100% of the subjects to which the antibody has been administered. The term, however, requires that the prevention occurs in a statistically significant portion of subjects (e.g. a cohort in a cohort study). Whether a portion is statistically significant can be determined without further ado by a person skilled in the art using various well-known statistic evaluation tools, e.g., determination of confidence intervals, p-value determination, Student's t-test, Mann-Whitney-U test etc. Details are described elsewhere herein.

The term “subject” as used herein relates to animals, preferably mammals, and, more preferably, humans. The subject according to the present invention shall be a subject suffering from or suspected to suffer from SARS-COV-2 infection. Typically, such a subject shows already symptoms associated with SARS-COV-2 infection or has been in contact with one or more other subjects known to suffer from SARS-COV-2 infection and, thus, is at risk of being infected as well.

The term “disease or condition referred to herein is associated with SARS-COV-2 infection” refers to any disease or condition resulting directly or indirectly from an infection by SARS-CoV-2. Preferably, the disease referred to herein is Covid 19. Moreover, the term also encompasses any symptom associated with SARS-COV-2 infection. Typically, such symptoms may be fever, cough, headache, fatigue, breathing difficulties, loss of smell, and loss of taste, respiratory failure, or multi-organ dysfunction.

Preferably, said disease or disorder is Covid-19 or any symptom associated therewith. Symptoms associated with Covid-19 are well known to the skilled artisan and may vary depending on the SARS-COV-2 variant concerned.

The present invention also relates to a method for treating and/or preventing disease or condition referred to herein is associated with SARS-COV-2 infection in a subject comprising administering to said subject a therapeutically effective amount of the modified ACE2 protein, the polynucleotide or the vector or expression construct of the invention.

It will be understood in accordance with the aforementioned therapeutic uses and methods of the invention that if the disease or disorder associated with SARS-COV-2 infection is associated with alveolar macrophage activation, the modified ACE2 protein or the polynucleotide or the vector or expression construct as defined in accordance with the present invention shall, preferably, have a modified Fc domain.

Moreover, it will be understood in accordance with the aforementioned therapeutic uses and methods of the invention that if the disease or disorder associated with SARS-COV-2 infection is not associated with alveolar macrophage activation, the modified ACE2 protein or the polynucleotide or the vector or expression construct as defined in accordance with the present invention shall, preferably, have a modified Fc+ domain.

Yet, the present invention relates to a kit comprising the modified ACE2 protein, the polynucleotide or the vector or expression construct of the invention.

The term “kit” as used herein refers to a collection of components comprising, inter alia, the modified ACE2 protein, the polynucleotide or the vector or expression construct of the invention in one or more container(s). The kit shall in addition to the modified ACE2 protein, the polynucleotide or the vector or expression construct of the invention also comprise further reagents. These reagents may include components that may be useful for facilitating uptake and/or integration of the modified ACE2 protein, the polynucleotide or the vector or expression construct of the invention. Moreover, such reagents may encompass any buffer solutions required as diluents. The container also typically comprises instructions for using the compounds in a subject or in ex vivo applications such as cell culture. These instructions may be in the form of a manual or may be provided by a computer program code.

The following are particular preferred embodiments of the invention.
Embodiment 1: A modified ACE2 protein having increased binding affinity for the SARS-CoV-2 spike protein compared to wildtype ACE2 protein, wherein said modified ACE2 protein is lacking enzymatic activity and is fused to:
(i) a modified Fc domain of human immunoglobulin, wherein the Fc domain has either enhanced immunostimulating activity (Fc⁺) or attenuated immunostimulating activity (Fc⁻); or
(ii) a protein that specifically binds to T cells or further enhances immunostimulating activity.
Embodiment 2: The modified ACE2 protein of embodiment 1, wherein said increased binding affinity for the SARS-COV-2 spike protein is increased binding affinity for the receptor binding domain (RBD) of the SARS-COV-2 spike protein
Embodiment 3: The modified ACE2 protein of embodiment 1 or 2, wherein said SARS-COV-2 spike protein is a wildtype SARS-COV-2 spike protein or a variant thereof, preferably, a spike protein of SARS-COV-2 alpha, beta, gamma, delta, kappa or omicron including its sub-variants.
Embodiment 4: The modified ACE2 protein of any one of embodiments 1 to 3, wherein said ACE2 protein comprises an amino acid sequence selected from the group consisting of:
a) an amino acid sequence as shown in SEQ ID NO: 3, 4, 6 or 8;
b) an amino acid sequence having at least 70% sequence identity to the amino acid sequence shown in SEQ ID NO: 3, 4, 6 or 8;
c) an amino acid sequence of b) which has in its amino acid sequence the following amino acids: T27Y, L79Y, N330Y; and
d) an amino acid sequence of b) or c) which has in its amino acid sequence at least one of the following further amino acids: K31Y, H34V, E35Q, or M82R.
Embodiment 5: The modified ACE2 protein of any one of embodiments 1-4, wherein said modified Fc domain has a reduced binding capability for the Fc receptor.
Embodiment 6: The modified ACE2 protein of any one of embodiments 1-4, wherein said modified Fc domain has an increased binding capability for the Fc receptor, preferably, comprises the SDIE modification.
Embodiment 7: A polynucleotide encoding the modified ACE2 protein of any one of embodiments 1 to 6.
Embodiment 8: A vector or expression construct comprising the polynucleotide of embodiment 7.
Embodiment 9: A host cell comprising the polynucleotide of claim 7 or the vector of embodiment 8.
Embodiment 10: A non-human transgenic organism comprising the polynucleotide of embodiment 7 or the vector or expression construct of embodiment 8.
Embodiment 11: A method for the manufacture of a modified ACE protein of any one of embodiments 1 to 6 comprising the steps of:
   (a) expressing the polynucleotide of embodiment 7 or the vector or expression construct of embodiment 8 in a host cell; and
   (b) obtaining the modified ACE2 protein from said host cell.
Embodiment 12: A medicament comprising the modified ACE2 protein of any one of embodiments 1 to 6, the polynucleotide of embodiment 7 or the vector or expression construct of embodiment 8.
Embodiment 13: A modified ACE2 protein as defined in any one of embodiments 1 to 6, a polynucleotide as defined in embodiment 7 or a vector or expression construct as defined in embodiment 8 for use in treating and/or preventing a disease or disorder associated with SARS-CoV-2 infection.
Embodiment 14: The modified ACE2 protein, the polynucleotide, the vector or expression construct for use of embodiment 13, wherein said disease or disorder is Covid-19 or any symptom associated therewith.
Embodiment 15: The modified ACE2 protein, the polynucleotide, the vector or expression construct for use of embodiment 13 or 14, wherein if the disease or disorder associated with SARS-COV-2 infection is associated with alveolar macrophage activation, the modified ACE2 protein or the polynucleotide or the vector or expression construct shall have a modified Fc-domain.
Embodiment 16: The modified ACE2 protein, the polynucleotide, the vector or expression construct for use of embodiment 13 or 14, wherein if the disease or disorder associated with SARS-COV-2 infection is not associated with alveolar macrophage activation, the modified ACE2 protein or the polynucleotide or the vector or expression construct shall have a modified Fc+ domain.
Embodiment 17: A kit comprising the modified ACE2 protein of any one of embodiments 1 to 6, the polynucleotide of embodiment 7 or the vector or expression construct of embodiment 8.
Embodiment 18: A method for treating and/or preventing a disease or disorder associated with SARS-COV-2 infection in a subject in need thereof comprising administering a therapeutically effective dosage of an modified ACE2 protein as defined in any one of embodiments 1 to 6, a polynucleotide as defined in embodiment 7 or a vector or expression construct as defined in embodiment 8 to said subject.
Embodiment 19: The method of claim 18, wherein if the disease or disorder associated with SARS-COV-2 infection is associated with alveolar macrophage activation, the modified ACE2 protein or the polynucleotide or the vector or expression construct shall have a modified Fc-domain.
Embodiment 20: The method of claim 18, wherein if the disease or disorder associated with SARS-COV-2 infection is not associated with alveolar macrophage activation, the modified ACE2 protein or the polynucleotide or the vector or expression construct shall have a modified Fc+ domain.
Embodiment 21: The method of any one of embodiments 18 to 20, wherein said disease or disorder is Covid-19 or any symptom associated therewith.

All references cited throughout this specification are herewith incorporated by reference in their entirety as well as with respect to the specifically mentioned disclosure content.

### FIGURES

FIG. 1: Construction and biochemical characterization of various ACE2 fusion proteins. (A) All depicted fusion proteins contain the peptidase- and collectrin domains of the ACE2 protein, a hinge region and a previously developed version of a human IgG1 Fc part that does no longer bind to human Fc receptors type I, II, and III (Fc). Additional mutations were introduced into the ACE2-part of the fusion protein to deplete its enzymatic activity (ACE2-RR-Fc) and to improve binding to the spike protein of the SARSCov2 virus (ACE2-M1-Fc and ACE2-M2-Fc). (B) and (C) depict the analysis of the proteins by SDS PAGE electrophoresis and size exclusion chromatography, respectively, after purification by protein A chromatography from the supernatant of transiently transfected CHO cells.

FIG. 2: Enzymatic activity of the fusion proteins. The catalytic activity of the ACE2 fusion proteins was measured using the ACE2 fluorometric assay kit (BioVision). This kit utilizes the ability of active ACE2 to cleave a synthetic MCA-peptide substrate to release a free fluorophore. The released MCA was quantified using a fluorescence microplate reader (EnVision Multilabel Counter). All proteins were diluted in the assay buffer to a final concentration of 90, 45, 22.5, 4.5 and 0.9 nM according to the manufacturer's instructions. The results shown were obtained with 4.5 nM ACE2 proteins.

FIG. 3: Inhibition of ACE2 binding to various recombinant spike proteins by fusion proteins and a reference antibody. (A)-(F) The indicated competing proteins were mixed with 150 nM His tagged ACE2 fusion protein (Biolegend) and added for 1 hour at room temperature to a 96 well ELISA plate pre-coated (overnight at 4° C.) with 1 μg/ml of full-length trimeric spike proteins of different viral variants as indicated. The plates were washed and incubated first with a Penta-anti-His conjugate (Qiagen) and then with HRP-streptavidin. After washing, TMB substrate was added. (G) Calculated IC₅₀ values.

FIG. 4: Binding of different ACE2 fusion proteins and the REGN10933 reference antibody to virus infected cells. ACE2 proteins were incubated at the indicated concentrations with Caco-2 cells infected with the original version of SARS-COV-2 (4° C. for 1 hour). Cells were then incubated with a PE-conjugated goat anti-human Fc (Jackson ImmunoResearch) and analyzed by flow cytometry.

FIG. 5: Neutralization of the original and the beta-variant of SARS-COV-2 by the ACE2 fusion proteins and a reference antibody. Caco-2 cells were infected with the original version (A) or the beta variant (B) of SARS-COV-2 and infected cells were incubated with the reagents indicated. After 48 h cells were fixed and immunofluorescence was performed using an Alexa-conjugated antibody targeting SARS-COV-2 NC. The infection rate was calculated as a number of infected cells (Alexa positive cells)/total amount of cells (DAPI positive cells).

FIG. 6: Neutralization of the original and the Omicron-variant of SARS-COV-2 by the ACE2 fusion proteins and a reference antibody. Caco-2 cells were infected with the original version (A) or the Omicron variant (B) of SARS-COV-2 and infected cells were incubated with the reagents indicated. After 48 h cells were fixed and immunofluorescence was performed using an Alexa-conjugated antibody targeting SARS-COV-2 NC. The infection rate was calculated as a number of infected cells (Alexa positive cells)/total amount of cells (DAPI positive cells).

FIG. 7: Amino acid sequences of ACE2 constructs. (A) ACE2-FcKO, (B) ACE2-RR-FcKO, (C) ACE2-M1-FcKO, (D) ACE2-M2-FcKO, (E) ACE2-M1-FcWT, (F) ACE2-M1-SDIE, (G) ACE2-M2-FcWT, (H) ACE2-M2-SDIE.

FIG. 8: Binding of ACE2-M1-FcKO fusion proteins and CD20-SDIE reference antibody to Fc receptors. Binding of ACE2-M1-FcKO and a CD20 antibody carrying SDIE mutations to the indicated His-tagged FcR proteins was determined by ELISA. Results represent means of two independent experiments.

FIG. 9: NK cell activation and cytokine release evaluation of ACE2-M1 fusion proteins. Omicron trimeric S-protein was coated in 96-well plates. PBMCs from healthy donors (n=2) and the indicated constructs at two different concentrations (50 nM and 20 nM) were added to the coated plates. Three days later, NK cell activation was monitored by flow cytometry (A) and cytokine secretion in culture media was analyzed using the LEGENDplex™ multiplex kit (B).

Sequences referred to in the application:
SEQ ID NO: 1: Amino acid sequence of ACE2-FcKO
SEQ ID NO: 2: Amino acid sequence of ACE2-RR-FcKO
SEQ ID NO: 3: Amino acid sequence of ACE2-M1-FcKO
SEQ ID NO: 4: Amino acid sequence of ACE2-M2-FcKO
SEQ ID NO: 5: Amino acid sequence of ACE2-M1-FcWT
SEQ ID NO: 6: Amino acid sequence of ACE2-M1-SDIE
SEQ ID NO: 7: Amino acid sequence of ACE2-M2-FcWT
SEQ ID NO: 8: Amino acid sequence of ACE2-M2-SDIE
FcWT: wildtype Fc domain
FcKO: attenuated Fc domain (Fc−)
ACE2: wildtype ACE2
ACE2-RR: enzymatic depleted ACE2
ACE2-M1: enzymatic depleted ACE2 with improved spike protein binding
ACE2-M2: enzymatic depleted ACE2 with improved spike protein binding
SDIE: Fc domain with SDIE modification (Fc+)

### EXAMPLES

The invention will be illustrated by the following Examples. The Examples shall not be construed as limiting the scope of the invention.

### Example 1. Generation and Production of ACE2 Fusion Proteins and REGN10933

Human ACE2 extracellular domain (aa 18-740; Gene ID: 59272) and the variable domain sequences of the REGN10933 antibody were codon optimized for Chinese hamster using the GeneArt GeneOptimizer tool (Thermo Fisher Scientific, Regensburg, Germany). VH, VL, and ACE2 sequences (ACE2 wild-type or the indicated mutants ACE2-RR, ACE2-M1 and ACE2-M2) were synthesized de novo at GeneArt (Thermo Fisher Scientific, Regensburg, Germany). ACE-2 coding sequences were fused at their C-terminus to a human Igγ1 Hinge-Fc domain via a flexible (GGGGS) 3 linker (FcWT). Modifications in the CH2 domain consisting of the amino acid substitutions and deletions E233P; L234V; L235A; ΔG236; D265G; A327Q; A330S (EU index), which abrogate FcR binding and complement fixation (FcKO), or S239D; 1332E (EU index) modifications to increase FcR binding (SDIE) were introduced as described.

REGN10933 variable sequences were inserted into a human Igγ1 backbone comprising CH1-CH2-CH3- or CK-constant domain sequences. All constructs were transiently transfected and produced using the ExpiCHO™ Expression System (Thermo Fisher Scientific, Regensburg, Germany) according to the manufacturer's instructions and were then purified by HiTrap™ MabSelect™ SuRe columns (Cytiva, Freiburg, Germany), before being subjected to preparative and analytical size exclusion chromatography (SEC) using HiLoad™ 16/600 Superdex 200 μg and Superdex™ 200 Increase 10/300 GL columns (Cytiva Freiburg, Germany), respectively. Endotoxin levels of samples, as determined by a limulus amebocyte lysate assay (Endosafe®Charles River, Charleston, SC), were <0.5 EU/ml. Sodium dodecyl sulfate-polyacrylamide gel electrophoresis (SDS-PAGE) was performed.

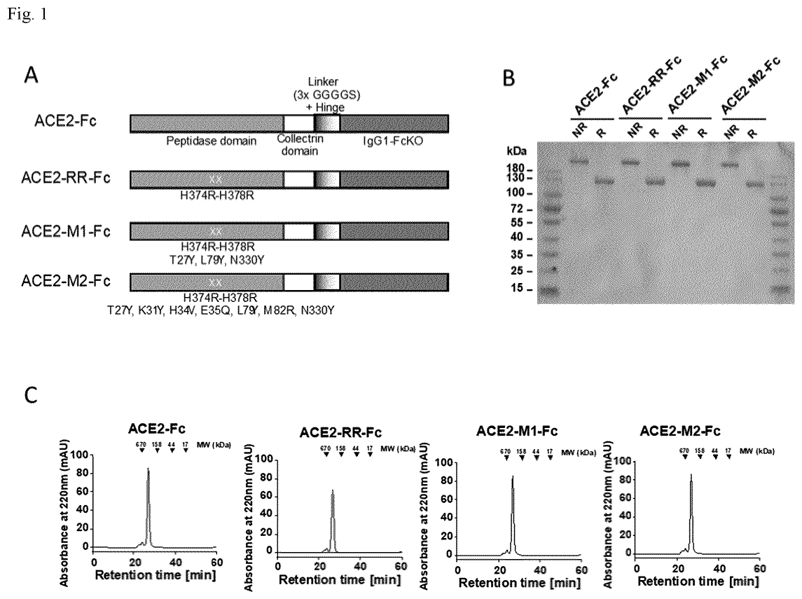

All fusion proteins are purified as stable homodimers from the supernatant of production cells transiently transfected with the respective genetic constructs (FIG. 1).

### Spike Proteins

SARS-COV-2 full-length trimeric spike proteins corresponding to the parental, Alpha (B.1.1.7), Beta (B1.351), Gamma (P.1) and Delta (B.1.617.2) were purchased from BioServ, (Sheffield, UK). The Trimeric S-protein corresponding to Omicron (B.1.1.529) from Sino Biological (Bejing, China).

### Example 2. ACE2 Catalytic Activity Assay

Enzymatic activity of ACE2 fusion proteins was measured using the ACE2 Activity Assay Kit (Fluorometric) (BioVision, Milpitas, CA) according to the manufacturer's instructions. The proteins were diluted in assay buffer to 22.7, 4.54 and 0.91 nM final concentration. Fluorescence was measured using a Wallac 1420 Victor 2 Multi-Label Counter (Perkin-Elmer, Waltham, MA).

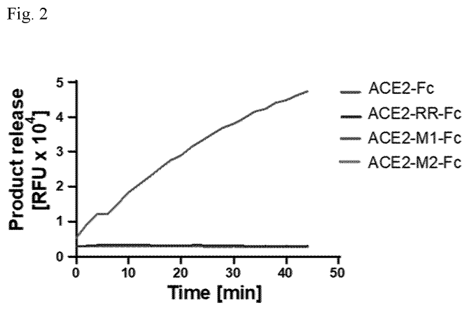

The wildtype fusion protein ACE2-Fc was enzymatically active, the three mutated proteins were completely devoid of ACE2 enzymatic activity (FIG. 2).

### Example 3. Competitive ELISA

For this assay, the indicated S trimeric proteins were coated on 96-well plates at 1 μg/ml, 4° C. overnight. After washing, wells were blocked with PBS-3% BSA for 1 hour at room temperature. Next, a serial dilution of the indicated ACE2 fusion proteins, REGN10933 or serum antibodies were pre-mixed with 150 nM of His-tagged ACE2 wild-type protein (BioLegend, San Diego, CA) and added to the plates. In case of the Omicron variant (Sino Biological), the His-tagged ACE2 protein was additionally biotinylated using the One-Step Biotinylation Kit (Miltenyi, Cologne, Germany) according to the manufacturer's instructions. For visualization, Penta-His HRP conjugate (1:1000) (Qiagen, Hilden, Germany) or mouse anti-Biotin HRP conjugate (1:1000) (Invitrogen, Waltham, MA) were used. Unbound HRP-conjugated antibodies were removed by washing, TMB substrate was added, and absorbance was measured at 450 nm.

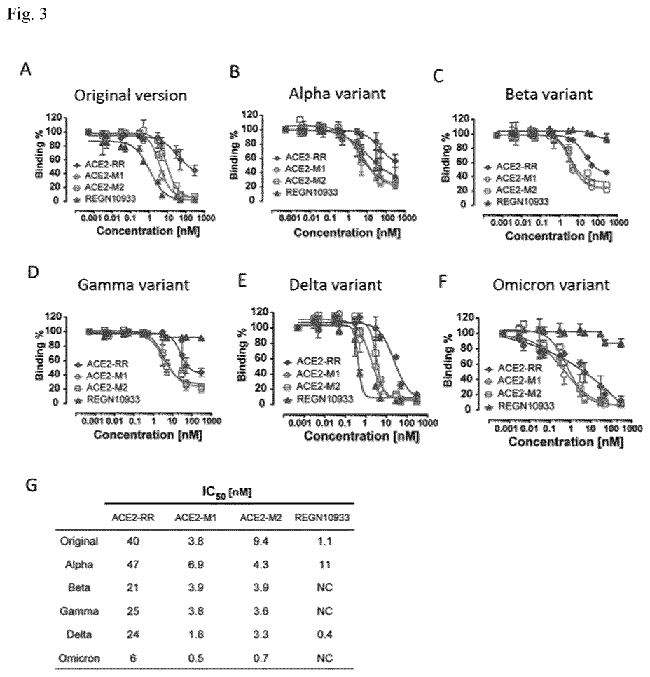

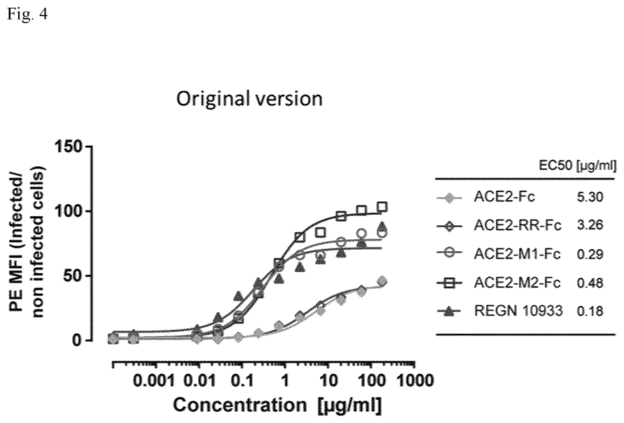

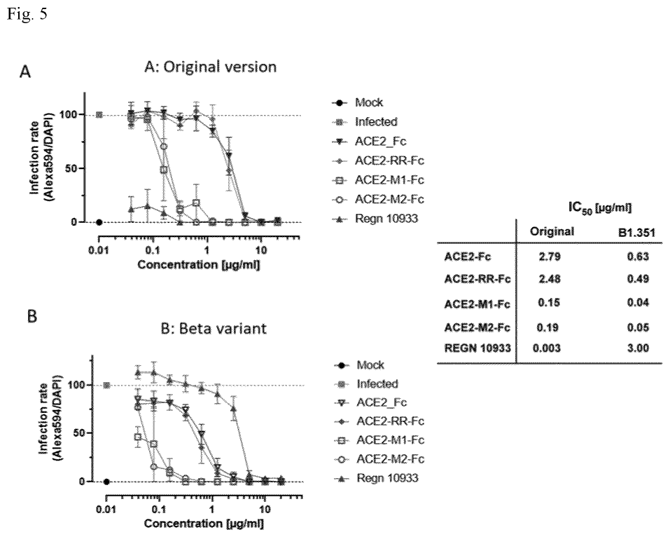

Binding of the affinity-mutated fusion protein versions M1 and M2 to all variants of the spike protein is improved if compared to the RR version. Binding of the fusion proteins to variant spike proteins (compared to the original version) is preserved or increased, whereas binding of the reference antibody to variant proteins is decreased or completely abrogated (FIG. 3).

### Example 4. Binding of ACE2 Fusion Proteins to SARS-COV-2 Infected Cells

For binding experiments, 3×10⁶ Caco-2 cells were seeded in a T75 flask the day before infection, in a medium containing 5% FCS. Cells were infected with SARS-COV-2-mNG, and 48 hpi cells were detached from the flask using Accutase, fixed with 2% PFA for 10 minutes at 37° C., and resuspended in FACS buffer (PBS, 1% FCS). 1×10⁶ cells in 100 μL in FACS buffer were distributed in a U-shape 96-well plate. The plate was centrifuged at 600 g for 5 min and the buffer was removed by a fast decant. Cells were incubated for 1 h at 4° C. using 50 μl of 3-fold serial dilutions of ACE2 protein or Regeneron (company), tested from 40 μg/ml following 12 dilution points. Cells were washed with 150 μl of FACS buffer/well, centrifuged, and the supernatant decanted. The washing step was repeated using 200 μl of FACS buffer/well. Subsequently, cells were incubated with 50 μl of a 1:200 dilution of the Secondary AB- R-Phycoerythrin (PE) conjugated affinity pure F(ab′)2 Fragment Goat-anti Human IgG-Fc gamma fragment (Jackson-Immuno) for 30 minutes at 4°. The two washing steps were repeated, and the cells were resuspended using 100 μl of FACS buffer/well. Controls included: mock-infected cells incubated with the highest and lower protein concentrations; infected cells non-incubated as well as infected cells stained only with the secondary antibody.

Binding of the affinity-mutated fusion protein versions M1 and M2 is comparable to that of the reference antibody and markedly improved compared to the RR- and the wildtype version of the ACE2 fusion protein. This difference is more pronounced if binding to infected cells is measured (rather than to recombinant proteins as depicted in FIG. 3).

### Example 5. Virus Neutralization Assay

### Viruses

All experiments with SARS-COV-2 viruses were conducted in a Biosafety Level 3 laboratory at the University Hospital Tübingen. The SARS-COV-2 strain icSARS-COV-2 was obtained from the World Reference Center for Emerging Viruses and Arboviruses (WRCEVA) of the UTMB (University of Texas Medical Branch, Galveston, TX, USA). SARS-COV-2 B.1 (SARS-CoV-2 Tü1), SARS-COV-2 B.1.351 (Beta) and SARS-COV-2 B.1.1.529 (Omicron) were isolated from patient samples and variant identity was confirmed by next-generation sequencing of the entire viral genome as described before. All virus stocks were generated in Caco-2 cells collecting supernatants 48-72 h post-infection (hpi). Multiplicity of infection determination (MOI) was conducted by titration using serial dilutions of both virus stocks. The number of infectious virus particles per millimeter was calculated as (MOI×cell number)/(infection volume), where MOI=_ln (1_infection rate).

Caco-2 (Human Colorectal adenocarcinoma, ATCC HTB-37) cells were cultured at 37° C. with 5% CO₂ in DMEM containing 10% FCS, 2 mM L-glutamine, 100 mg/ml penicillin-streptomycin and 1% NEAA. Neutralization assays using clinical isolates were performed as described in Wagner et al., 2021. Briefly, cells were co-incubated with the clinical isolate SARS-COV-2 B.1, SARS-COV-2 B.1.351 (Beta) or SARS-COV-2 B.1.1.529 (Omicron), at MOI of 0.7-1, and serial dilutions of the ACE2 protein designs from 20 to 0.039 μg/ml. 48 hpi, cells were fixed with 80% acetone, and immune fluorescence (IF) staining was performed using an anti-SARS-COV-2 nucleocapsid antibody (GeneTex, Cat No. GTX135357) and goat anti-rabbit Alexa594-conjugated secondary antibody. Cells were counterstained with DAPI solution and images were taken with the Cytation3 (BioTek). Infection rates were calculated as the ratio of DAPI-positive over Alexa594-positive cells, which were automatically counted by the Gen5 software (BioTek). Inhibitory concentration 50 (IC50) was calculated as the half-maximal inhibitory dose using four-parameter nonlinear regression (GraphPad Prism).

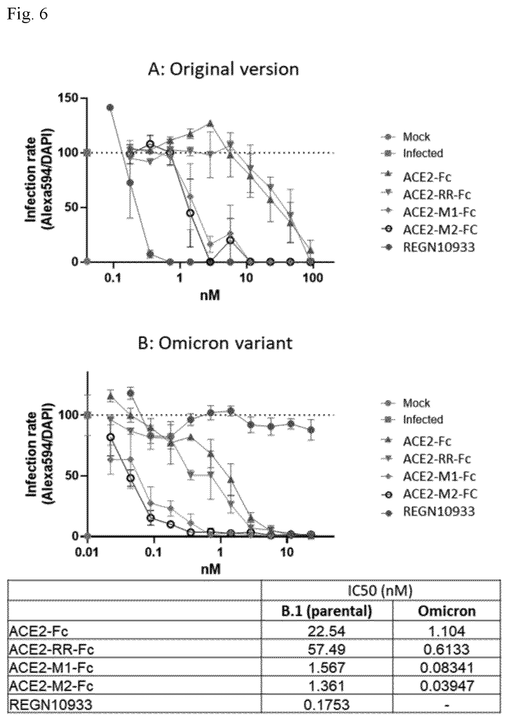

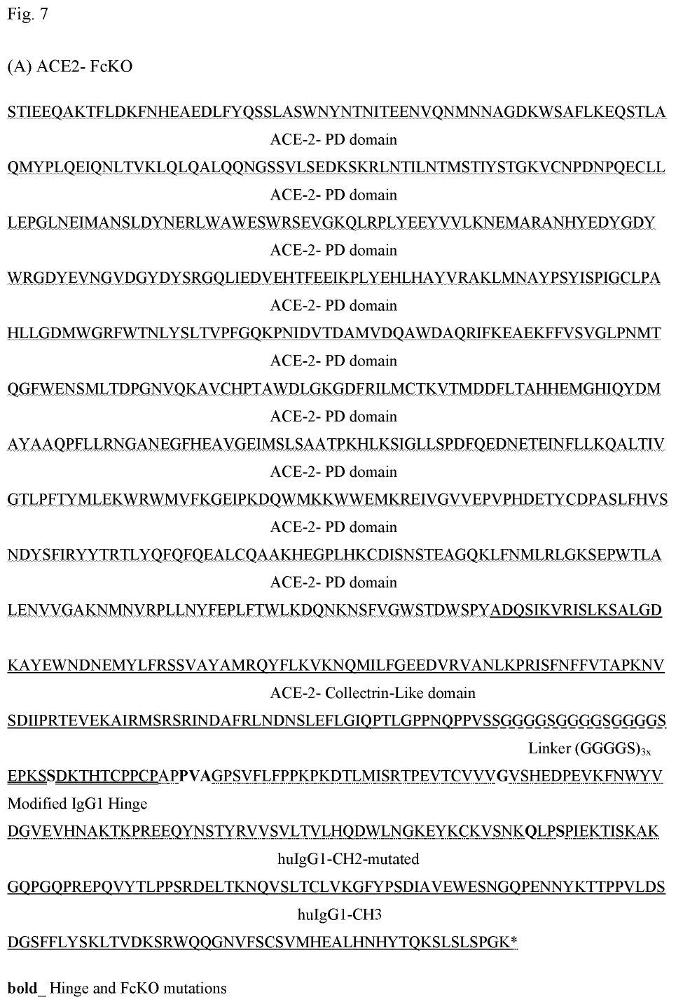

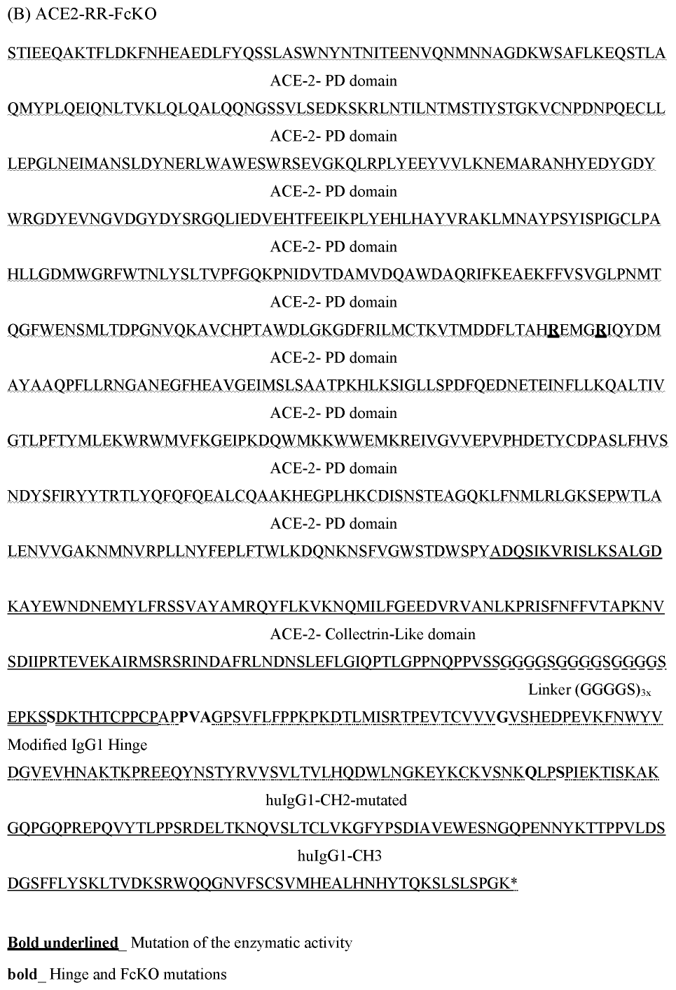

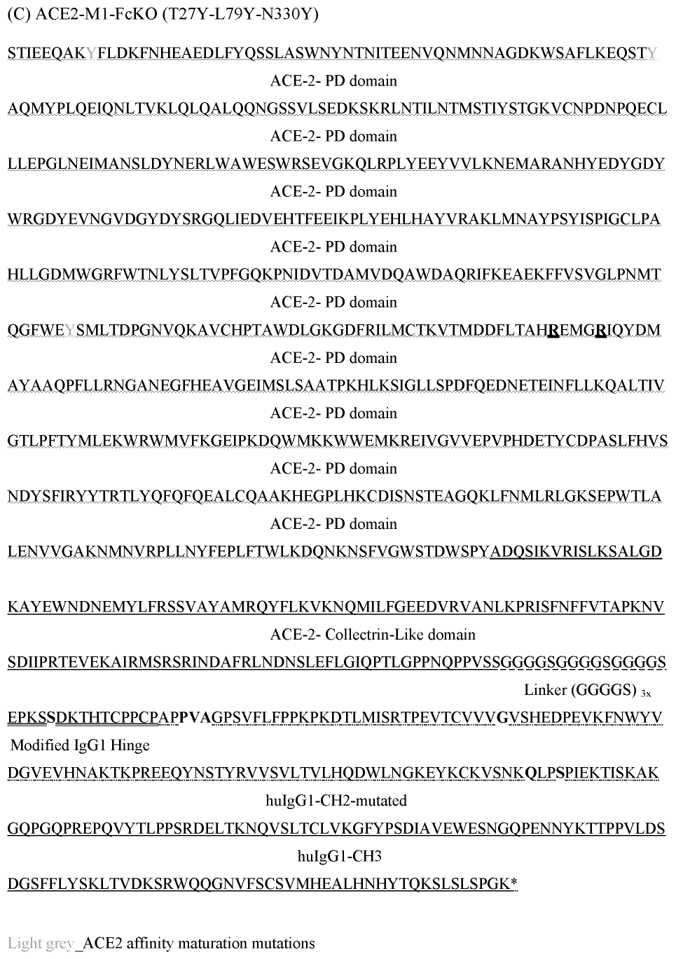

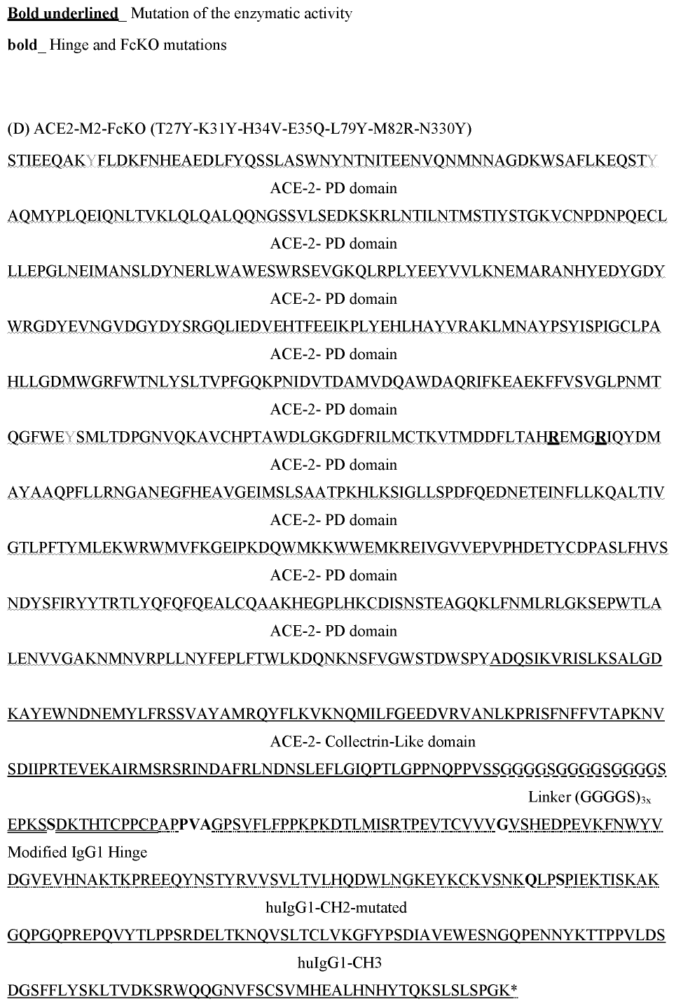

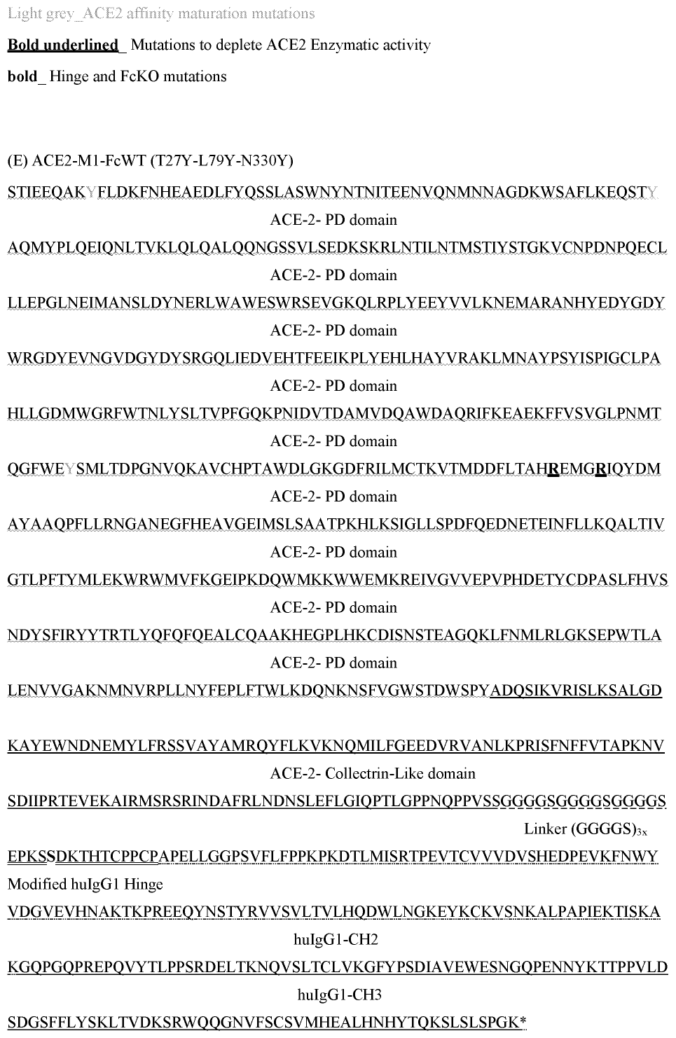

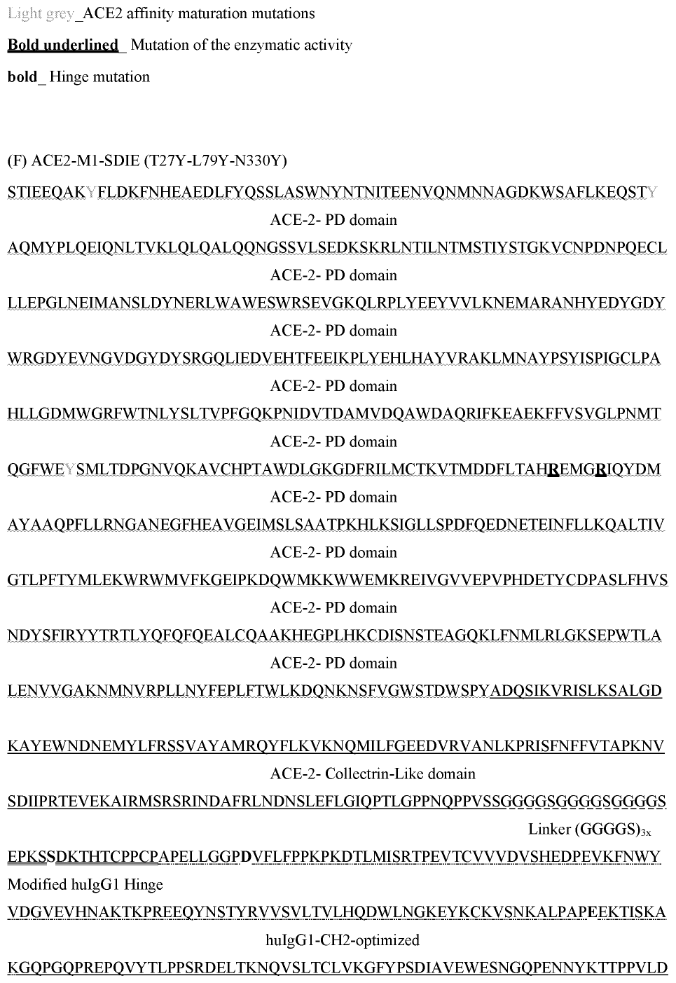

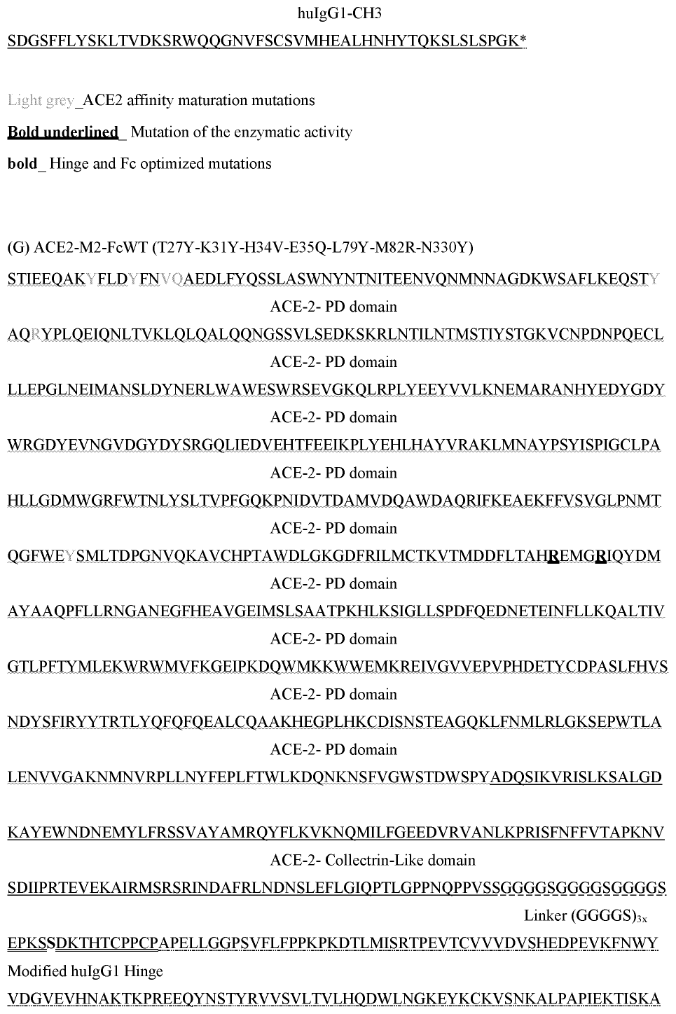

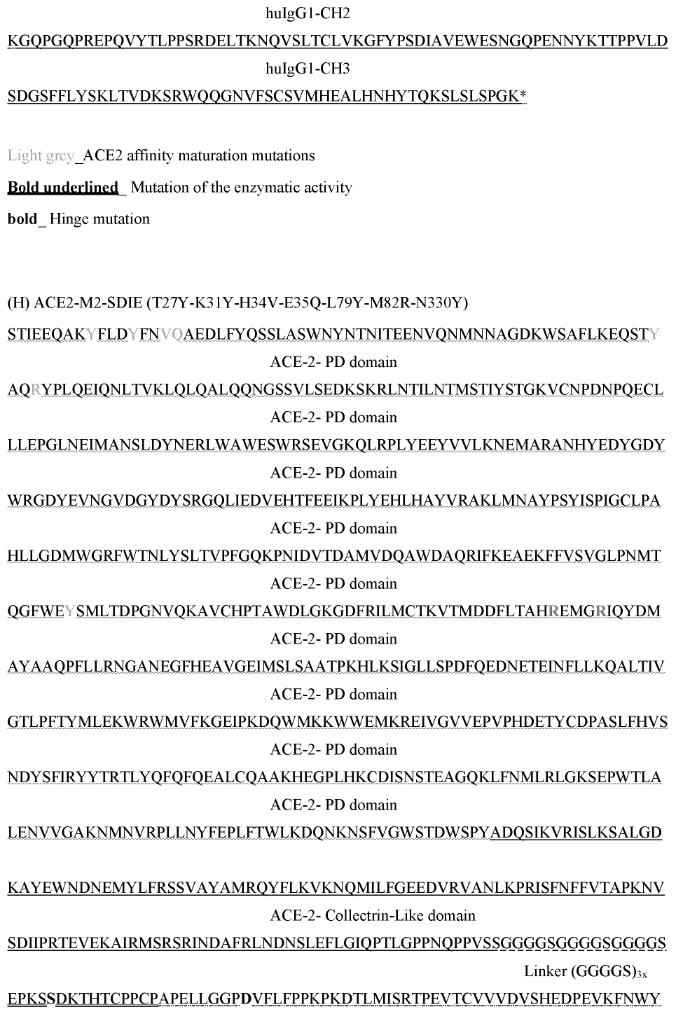

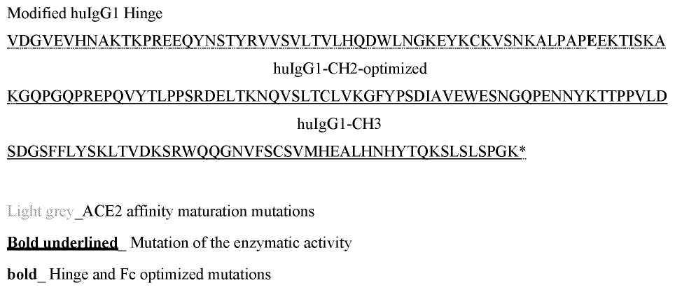

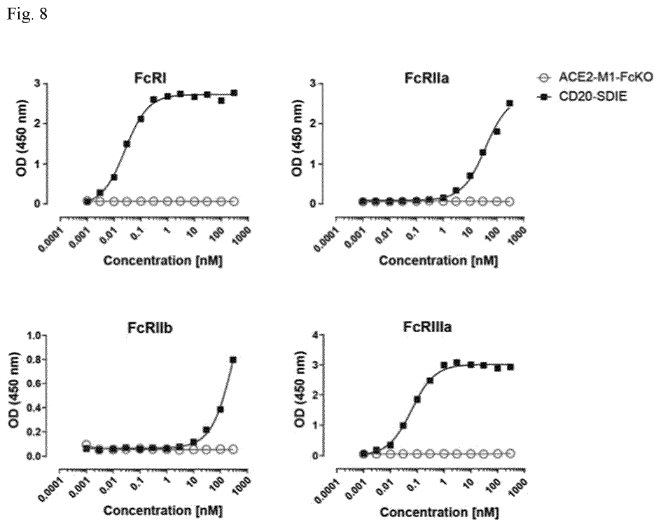

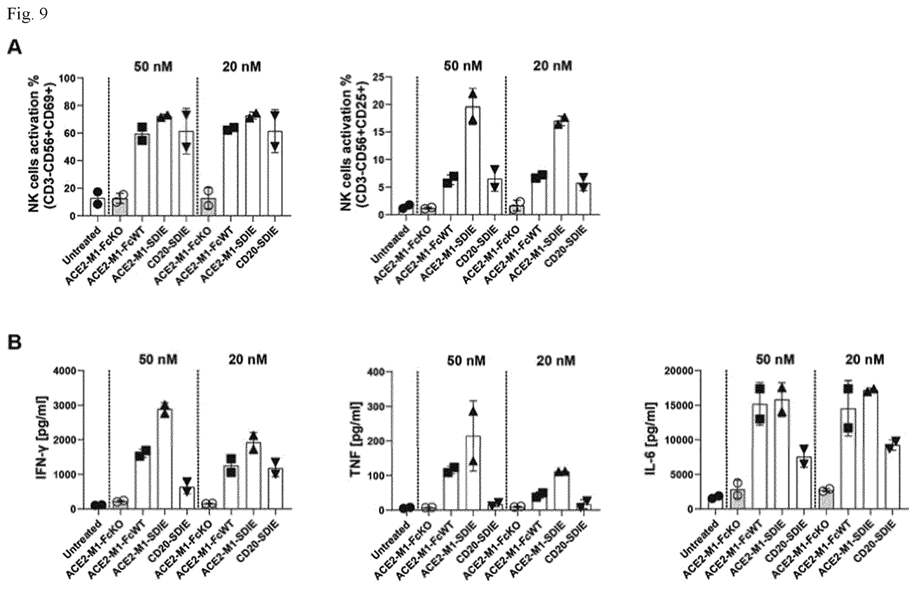

The neutralizing activity of the affinity-mutated M1 and M2 versions is markedly enhanced if compared to the—Fc and RR-Fc versions. The neutralizing activity of the fusion proteins against the beta variant is better than that against the original version, for the reference antibody REGN10933 it is much worse (FIG. 5). Against the Omicron variant, an even more enhanced neutralizing activity of M1 and M2 versions could be demonstrated, whereas the REGN10933 antibody was completely inactive. M2 versions showed an even better neutralizing activity than M1 versions (FIG. 6).

### Example 6. Fc Receptor Binding Assay

FcR binding analysis was conducted using ELISA by coating wells with his-tagged FcγRI, FcγRIIa, FcγRIIb, or FcRIIIa proteins (R&D Systems, Minneapolis, MN, USA). ACE2-M1-FcKO fusion protein or CD20-SDIE antibody (used as a positive control) were added to the plate at the indicated concentrations, and binding was visualized using an HRP-conjugated goat anti-human-Fc antibody (Jackson ImmunoResearch, West Grove, PA, USA). Unbound HRP-conjugated antibodies were removed by washing, TMB substrate was added, and absorbance was measured at 450 nm.

No significant binding was observed for ACE2-M1-FcKO fusion protein compared to the positive control demonstrating that the Fc activity of the ACE2-M1-FcKO fusion protein can be efficiently attenuated.

### Example 7. ACD2-Mediated Activation Assay

The Trimeric S-protein corresponding to Omicron (B.1.1.529) from Sino Biological (Bejing, China) was coated on 96-well plates at 5 μg/ml, 37° C. for 2 h. After washing, Peripheral Blood Mononuclear Cell (PBMCs) from healthy donors were incubated in the presence of the various constructs and incubated for 3 days at 37° C.-5% CO₂. Activated NK cells were defined as CD3-CD56+CD69+ or CD3 CD56 CD25+ cells using CD3, CD56, CD69, CD25 fluorescence-conjugates (Biolegend). Absolute cell numbers were calculated using equal numbers of BDTMCompBead (BD Biosciences). Dead cells were excluded using 7-AAD (BioLegend).

NK-cell activation was strongest for the ACE2-M1-SDIE fusion protein followed by ACE2-M1-FcWT fusion protein, while ACE2-M1-FcKO fusion protein showed reduced NK cell activation. Accordingly, the SDIE mutation in the Fc part when included into the fusion protein of the invention was demonstrated to enhance NK-cell activation, while a FcKO fusion protein of the invention exhibited reduced NK-cell activation potential. CD20-SDIE served as a (positive) control. Accordingly, it could be demonstrated that ACE2 fusion proteins with enhanced or reduced Fc activity could be provided.

### Example 8. Cytokine Measurements

For analysis of cytokine secretion, coated trimeric Omicron S-protein, PBMCs and ACE2 constructs were incubated for 72 h and culture supernatant were harvested. Cytokines were analyzed using the LEGENDplex™ multiplex kits (BioLegend) according to the manufacturer's instructions.

Strong cytokine secretion was shown for IFN-gamma, TNF and IL-6 for ACE2-M1-SDIE fusion protein followed by ACE2-M1-FcWT fusion protein while ACE2-M1-FcKO fusion protein showed reduced cytokine secretion. Accordingly, it could be further demonstrated that ACE2 fusion proteins with enhanced or reduced Fc activity could be provided.

### CITED LITERATURE

Asamow 2021, Cell 184, 3192-3204;
Sharma 2021, Proteins. 2021; 1-11;
WO2021/1207433
Saunders 2019, Front Immunol. 10:1296
U.S. Pat. No. 6,624,821
U.S. Pat. No. 5,624,821
WO2006/036291
WO2013/092001
WO2004/029207
Chan et al. 2020, Science 369:1261;
Higuchi et al. 2021, Nat. Commun. 12:3802
Payandeh et al. 2020, J. Theor. Biol. 505:110425
Ferrari et al. 2021, Journal of Virology, vol. 95, issue 19, e00685-21
Tanaka et al. 2021, Nature Scientific Reports, 11:12740
Svilenov et al., Antiviral Research 196, 105197
Kabat 1987, Sequences of Proteins of Immunological Interest (National Institute of Health, Bethesda, Md. 1987)
Riethmüller et al. 2012; Cancer Immun 12: 12
Neri D. 2019; Cancer Immun Res 7:348

1-14. (canceled)

15. A modified ACE2 protein having increased binding affinity for the SARS-COV-2 spike protein as compared to wildtype ACE2 protein, wherein said modified ACE2 protein is lacking enzymatic activity and is fused to:
(i) a modified Fc domain of human immunoglobulin, wherein the Fc domain has either enhanced immunostimulating activity (Fc⁺) or attenuated immunostimulating activity (Fc⁻); or
(ii) a protein that specifically binds to T cells or further enhances immunostimulating activity.

16. The modified ACE2 protein of claim 15, wherein said increased binding affinity for the SARS-COV-2 spike protein is increased binding affinity for the receptor binding domain (RBD) of the SARS-COV-2 spike protein

17. The modified ACE2 protein of claim 15, wherein said SARS-COV-2 spike protein is a wildtype SARS-COV-2 spike protein or a variant thereof, preferably, a spike protein of SARS-COV-2 alpha, beta, gamma, delta, kappa or omicron including its sub-variants.

18. The modified ACE2 protein of claim 15, wherein said modified ACE2 protein comprises an amino acid sequence selected from the group consisting of:
(a) an amino acid sequence as shown in SEQ ID NO: 3, 4, 6 or 8;
(b) an amino acid sequence having at least 70% sequence identity to the amino acid sequence shown in SEQ ID NO: 3, 4, 6 or 8;
(c) an amino acid sequence of b) which has in its amino acid sequence the following amino acids: T27Y, L79Y, N330Y; and
(d) an amino acid sequence of b) or c) which has in its amino acid sequence at least one of the following further amino acids: K31Y, H34V, E35Q, or M82R.

19. The modified ACE2 protein of claim 15, wherein said modified Fc domain has a reduced or increased binding capability for the Fc receptor.

20. The modified ACE2 protein of claim 15, wherein said modified Fc domain has an increased binding capability for the Fc receptor comprises the SDIE modification.

21. A polynucleotide encoding the modified ACE2 protein of claim 15.

22. A vector or expression construct comprising the polynucleotide of claim 21.

23. A host cell comprising the polynucleotide of claim 21.

24. A non-human transgenic organism comprising the polynucleotide of claim 21.

25. A method for the manufacture of a modified ACE protein of claim 15 comprising the steps of:
(a) expressing the polynucleotide of claim 21; and
(b) obtaining the modified ACE2 protein from said host cell.

26. A medicament comprising the modified ACE2 protein of claim 15.

27. A kit comprising the ACE2 protein of claim 15, the polynucleotide of claim 21.

28. A method for treating and/or preventing a disease or disorder associated with SARS-COV-2 infection in a subject in need thereof comprising administering a therapeutically effective dosage of an modified ACE2 protein as defined in claim 15, a polynucleotide as defined in claim 21 to said subject.

29. The method of claim 28, wherein if the disease or disorder associated with SARS-CoV-2 infection is associated with alveolar macrophage activation, the modified ACE2 protein shall have a modified Fc-domain.

30. The method of claim 28, wherein if the disease or disorder associated with SARS-CoV-2 infection is not associated with alveolar macrophage activation, the modified ACE2 protein shall have a modified Fc+ domain.

31. The method of claim 28, wherein said disease or disorder is Covid-19 or any symptom associated therewith.
